(19) United States
(12) Patent Application Publication
(10) Pub. No.: US 2025/0391495 A1
(43) Pub. Date: Dec. 25, 2025

(54) Deep Learning Enabled Prediction of Drug-Induced Liver Injury

(71) Applicant: Genentech, Inc., South San Francisco, CA (US)

(72) Inventors: Heng-Yi WU, Fremont, CA (US); Bingyuan CHEN, San Leandro, CA (US); Chloe Jinsol KIM, Redwood City, CA (US)

(21) Appl. No.: 19/306,210

(22) Filed: Aug. 21, 2025

Related U.S. Application Data
(63) Continuation of application No. PCT/US2024/016534, filed on Feb. 20, 2024.
(60) Provisional application No. 63/486,180, filed on Feb. 21, 2023.

(51) Int. Cl. G16B 5/20; G16B 40/20; G16H 70/40

(52) U.S. Cl. CPC G16B 5/20; G16B 40/20; G16H 70/40

(57) ABSTRACT

A method may include determining, based at least on a knowledge graph, a plurality of biological interaction profiles associated with a plurality of drugs. The knowledge graph being representative of a plurality of interactions between a variety of drugs, proteins, and a hierarchy of biological functions. Each biological interaction profile may be representative of the effects of a corresponding drug being propagated through protein-protein interactions and biological functions. A liver injury prediction model may be trained, based on a training dataset including the biological interaction profiles, a probability of drug induced liver injury. The liver injury prediction model to may be applied to determine, based on the biological interaction profile of a drug, the probability of liver injury associated with the drug. In some cases, the liver injury prediction model may further determine the probability of liver injury based on the molecular fingerprint and/or the molecular properties of the drug.

### PRIORITY

This application is a continuation, under 35 U.S.C. § 365(c), of International Patent Application No. PCT/US2024/016534, filed 20 Feb. 2024, which claims the benefit, under 35 U.S.C. § 119(e), of U.S. Provisional Patent Application No. 63/486,180, filed 21 Feb. 2023, each of which is incorporated herein by reference.

### TECHNICAL FIELD

The subject matter described herein relates generally to machine learning and more specifically to a deep learning-based technique for predicting the probability of drug-induced liver injury (DILI).

### BACKGROUND

Drug-induced liver injury (DILI) is a serious concern for patient safety and a major cause of drug candidate attrition and market withdrawal. Drug-induced liver injury is attributable to complicated intrinsic and idiosyncratic mechanisms. Intrinsic drug-induced liver injury refers to predictable and dose-dependent liver injury. Contrastingly, idiosyncratic drug-induced liver injury tend to be associated with host factors and individual susceptibility (e.g., gene variants, demographics, and/or the like) but is less contingent on the dose, route of administration, and duration of administration of the drug. In extreme cases, drug-induced liver injury may necessitate liver transplant or even cause death.

### SUMMARY OF PARTICULAR EMBODIMENTS

Systems, methods, and articles of manufacture, including computer program products, are provided for deep learning enabled prediction of drug-induced liver injury (DILI). Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the prediction of clinical outcomes in the context of liver injury engendered by exposure to certain small molecule drugs, it should be readily understood that such features are not intended to be limiting. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A depicts a system diagram illustrating an example of a liver injury prediction system, in accordance with some example embodiments;

FIG. 1B depicts a schematic diagram illustrating an example of a liver injury prediction model, in accordance with some example embodiments;

FIG. 1C depicts a schematic diagram illustrating an example of a liver injury prediction model, in accordance with some example embodiments;

FIG. 2A depicts a flowchart illustrating an example of a process for deep learning based prediction of drug-induced liver injury, in accordance with some example embodiments;

FIG. 2B depicts a flowchart illustrating another example of a process for deep learning based prediction of drug-induced liver injury, in accordance with some example embodiments;

FIG. 2C depicts a flowchart illustrating another example of a process for deep learning based prediction of drug-induced liver injury, in accordance with some example embodiments;

FIG. 3 depicts a schematic diagram illustrating an example of a multiscale interactome, in accordance with some example embodiments;

FIG. 4A depicts a schematic diagram illustrating an example of a process for generating the biological interaction profile of a drug, in accordance with some example embodiments;

FIG. 4B depicts a graph illustrating a relationship in the similarities between gene expression signature and biological interaction profile, in accordance with some example embodiments;

FIG. 4C depicts a graph illustrating a relationship between the performance of a liver injury prediction model and the quantity of dimensions in the biological interaction profile embedding used by the liver injury prediction model for determining the probability of drug-induced liver injury, in accordance with some example embodiments;

FIG. 5 depicts a schematic diagram illustrating an example of a process for generating the molecular fingerprint of a drug, in accordance with some example embodiments;

FIG. 6 depicts a schematic diagram illustrating an example of a process for generating training and testing data for a liver injury prediction model, in accordance with some example embodiments;

FIG. 7 depicts a block diagram illustrating an example of a computing system, in accordance with some example embodiments; and

FIG. 8 is an embodiment of a tSNE plot of diffusion profile embedding converted to vector space for a 500-bit vector.

Appendix A describes development of a multimodal learning model for drug development and drug-induced liver injury risk prediction,” the contents of which is incorporated herein by reference in its entirety.

When practical, similar reference numbers denote similar structures, features, or elements.

### DESCRIPTION OF EXAMPLE EMBODIMENTS

Due to the multitude of etiologies and multifactorial mechanisms associated with drug-induced liver injury, evaluating the risk of drug-induced liver injury (DILI) associated with a drug is a challenging task. Although a number of in vivo and in vitro approaches have been developed to assess the risk of drug-induced liver injury, these types of analysis are expensive, time-consuming, and unreliable. Table 1 depicts examples of conventional in silico techniques for evaluating the risk of liver injury associated with a drug. As shown in Table 1, conventional in silico techniques, including machine learning and deep learning-based methodologies, require drug-induced gene expression data in order to achieve adequate predictive performance. However, drug induced gene expression and assay data are scarce, thus imposing significant limits on the volume of samples available for the development and evaluation of existing in silico techniques for predicting the risk of drug-induced liver injury.

TABLE 1

| | | | DILI | |
|---|---|---|---|---|
| | | | Annotated | Performance |
| Method | Features | Model | data | (accuracy) |
| Graph Neural Network | Molecular | Graph Neural | Train/Test | ACC = 0.773 |
| for Drug-induced Liver | Fingerprints + | Network | with 483 | F-1 = 0.7883 |
| Injury Prediction using | Gene | | drugs from | |
| Transcriptome Data | Expression + | | LTKB | |
| (GLIT) (2020) | Pathway + | | Most-DILI- | |
| | Dose | | Concern: 114 | |
| | | | Less-DILI- | |
| | | | Concern: 268 | |
| | | | No-DILI- | |
| | | | Concern: 101 | |
| Predicting Drug- | Molecular | Ensemble | Train/Test | ACC = 0.711 |
| Induced Liver Injury | Fingerprints | model using 3 | with 1241 | Recall = 0.799 |
| Using Ensemble | | machine | drugs | |
| Learning Methods and | | learning | (SMILE | |
| Molecular Fingerprints | | algorithms | code) | |
| (2018) | | | DILI Positive: | |
| | | | 683 | |
| | | | DILI | |
| | | | Negative: 558 | |
| | | | External | |
| | | | drugs for | |
| | | | individual | |
| | | | validation: | |
| | | | from LTKB | |
| Deep Learning on | Gene | Deep Neural | Train/Test | ACC = 0.721 |
| High-Throughput | Expression | Network | with 1279 | Recall = 0.839 |
| Transcriptomics to | profiles | (DNN) model | drugs from | F = 1 = 0.795 |
| Predict Drug-Induced | | | drug-induced | |
| Liver Injury (2020) | | | liver injuryst | |
| DeepDILI: Deep | Molecular | Combined | Train/Test | ACC = 0.687 |
| learning-powered | Descriptors | machine | with 1002 | Recall = 0.805 |
| drug-induced liver | | learning (ML) | drugs from | F-1 = 0.755 |
| injury prediction using | | algorithms with | drug-induced | |
| model-level | | a deep learning | liver injuryst | |
| representation (2020) | | framework | DILI Positive: | |
| | | | 604 | |
| | | | DILI | |
| | | | Negative: 398 | |
| An in-silico model for | QSAR + FP2 | Machine | Train/Test | ACC = 0.730 |
| predicting drug- | fingerprints | learning | with 1458 | Recall = 0.773 |
| induced hepatotoxicity | | | from 14 data | |
| (2019) | | | sources. | |
| | | | DILI Positive: | |
| | | | 761 | |
| | | | DILI | |
| | | | Negative: 697 | |

Accordingly, in some example embodiments, a drug analysis engine may determine the risk of drug-induced liver injury without scarcely available data such as drug-induced gene expression data. For example, instead of drug-induced gene expression data for various biological targets of a drug (or drug molecule), the drug analysis engine may apply a liver injury prediction model to determine the probability of liver injury associated with the drug based on the molecular fingerprint of the drug, the biological interaction profile of the drug, and/or one or more molecular properties of the drug (e.g., molecular weight, topological surface area, partition coefficient (cLogP), distribution coefficient (cLogD), and/or the like). By leveraging the biological interaction profile of the drug, various implementations of the liver injury prediction model described herein is able to achieve comparable performance and, in instances where the biological interaction profile of the drug is further combined with its molecular fingerprint and molecular properties, superior performance than conventional techniques dependent on drug-induced gene expression data.

In some example embodiments, the drug analysis engine may generate, for the drug, a molecular fingerprint that encodes the molecular structure of the drug. For example, in some cases, the drug analysis engine may generate the molecular fingerprint to capture the structural similarities that may exist between different drugs. Moreover, in some cases, the molecular fingerprint of the drug may be an array of n elements, such as a first vector containing an n-bit long binary string corresponding to the molecular fingerprint of the drug. Accordingly, a similarity metric indicative of the structural similarity between two drugs (e.g., a Tanimoto index and/or the like) may be computed based a comparison of the respective molecular fingerprints of the two drugs. Examples of molecular fingerprints include deep learning based molecular fingerprints (e.g., sequence-based and geographic-based deep learning fingerprints) and rule-based molecular fingerprints (e.g., topological and circular topological fingerprints). For instance, in some cases, the drug analysis engine may determine, for the drug, an extended-connectivity fingerprint (ECFP), which is an example of a circular topological fingerprint. Furthermore, in some cases, the drug analysis engine may determine the molecular fingerprint (e.g., the extended-connectivity fingerprint (ECFP)) of the drug based on a computer-processable representation of the drug's molecular structure such as an isomeric Simplified Molecular Input Line Entry System (SMILES) code representation of the drug's molecular structure.

In some example embodiments, the drug analysis platform may generate, for the drug, a biological interaction profile representative of one or more effects of the drug being propagated through one or more protein-protein interactions and biological functions. In some cases, the biological interaction profile of the drug may be generated based on a knowledge graph representative of the various interactions between different drugs, proteins, and a hierarchy of biological functions. For example, the knowledge graph may include a network of interconnected nodes, each of which being representative of a drug, a protein, a biological function, or a disease. Moreover, each edge in the knowledge graph may be representative of a drug-protein interaction, a disease-protein interaction, a protein-protein interaction, a protein-biological function interaction, or a biological function-biological function interaction between the nodes in the knowledge graph connected by the edge. To generate the biological interaction profile of the drug, the drug analysis engine may traverse the knowledge graph. For instance, in some cases, the drug analysis engine may generate the biological interaction profile of the drug by performing one or more random walks across the knowledge graph. It should be appreciated that the biological interaction profile of the drug may also be known as (or referred to) as the diffusion profile of the drug.

In some example embodiments, each traversal of the knowledge graph may start from a node corresponding to the drug. In instances where the drug is new and a corresponding node is not present in the knowledge graph, each traversal of the knowledge graph may start from a node corresponding to a protein affected by the drug. Furthermore, each traversal of the knowledge graph may end at another node in the knowledge graph corresponding to a disease. Accordingly, in some cases, the biological interaction profile of the drug may include, for each node in the knowledge graph, a frequency of the node being visited during the one or more traversals of the knowledge graph. For example, in instances where the knowledge graph contains an m-quantity of nodes, the biological interaction profile of the drug may be an array of m elements, such as a second vector containing an m-quantity of values corresponding to the quantity of times each node of the m-quantity of nodes in the knowledge graph was visited during the one or more traversals of the knowledge graph. Moreover, in some cases, the first vector containing the n-bit long binary string corresponding to the molecular fingerprint of the drug may be concatenated with the second vector containing the m-quantity of values corresponding to the biological interaction profile of the drug as well as a third vector containing one or more values corresponding to the one or more molecular properties of the drug. The drug analysis engine may apply the liver injury prediction model to determine, based at least on a single vector formed by concatenating the three aforementioned vectors, to determine the probability of liver injury associated with the drug.

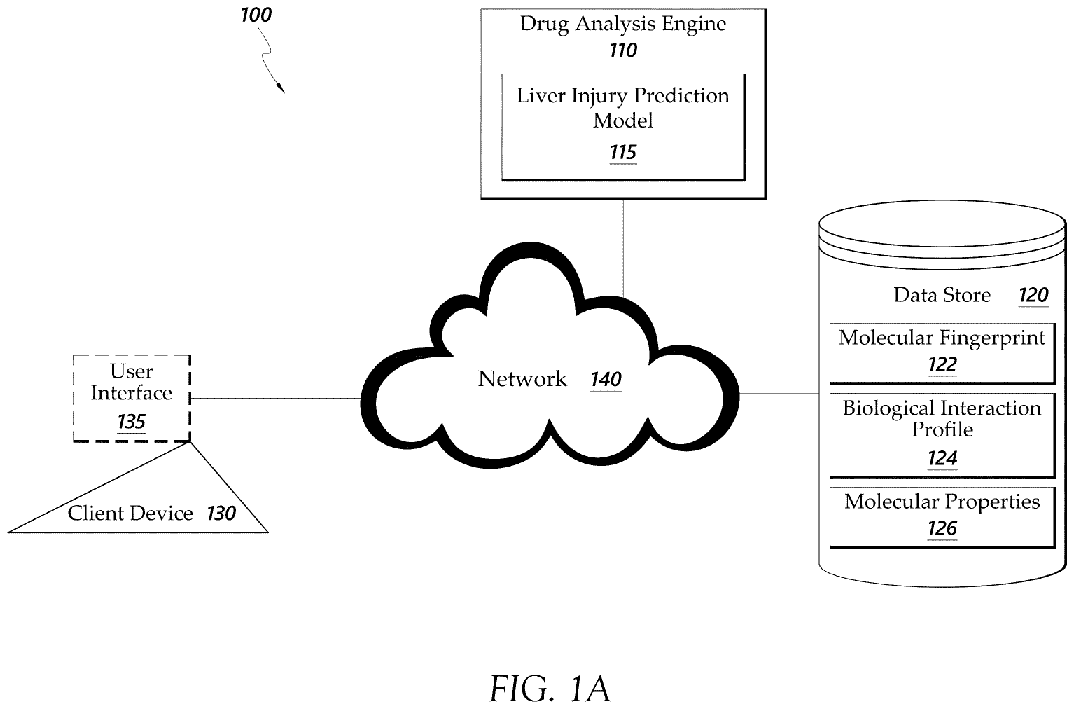

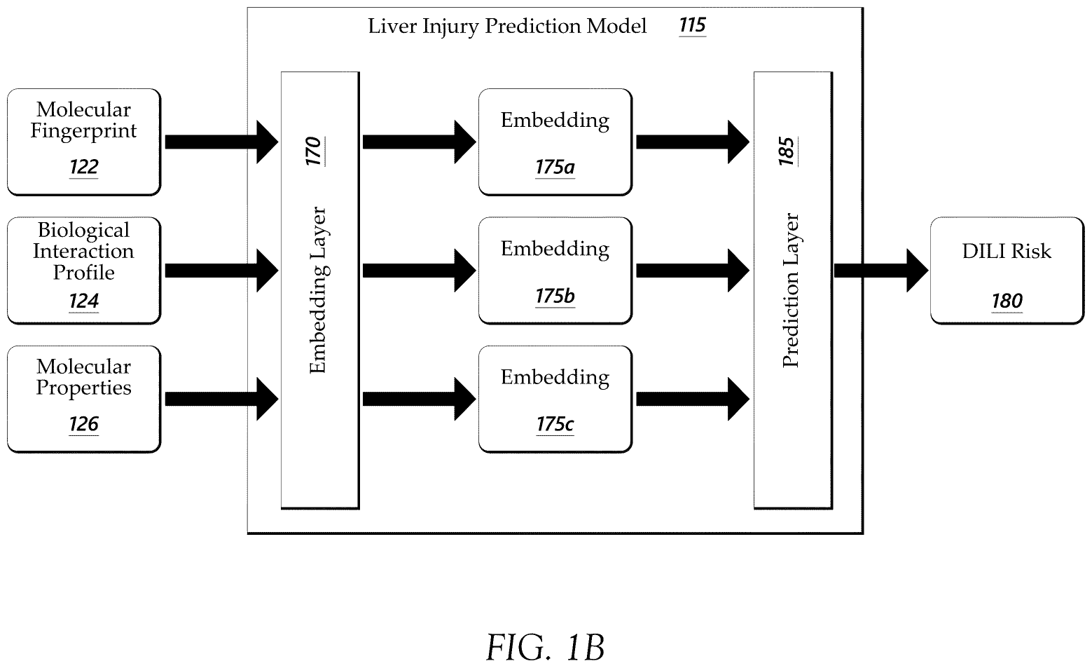

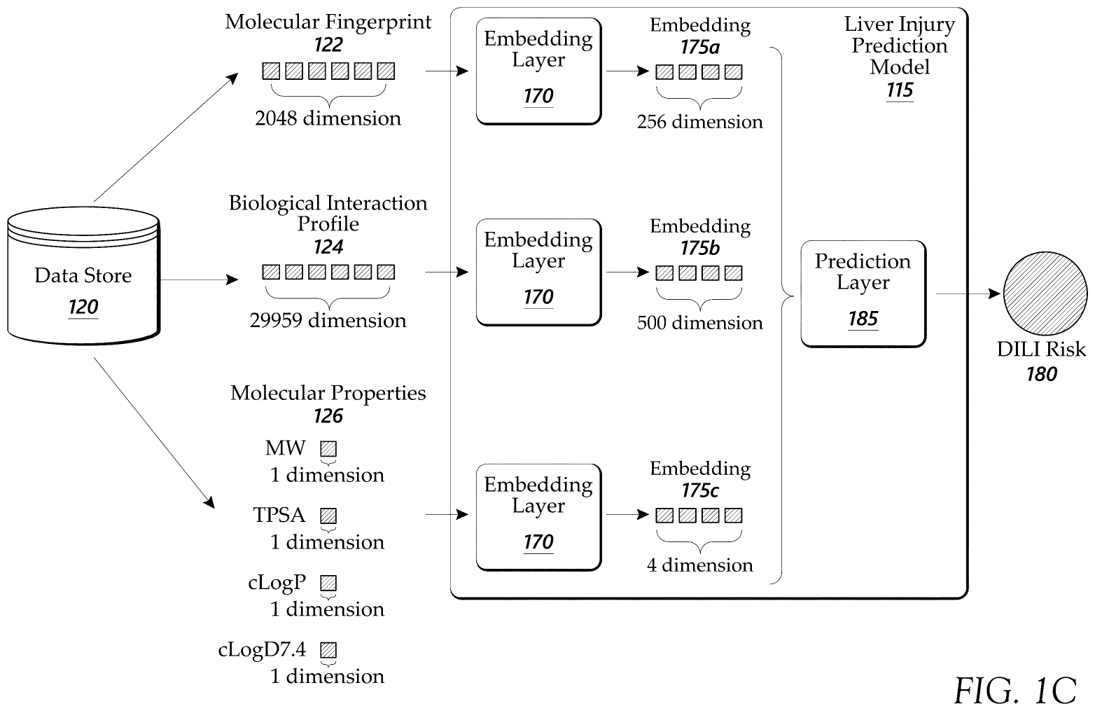

FIG. 1A depicts a system diagram illustrating an example of a liver injury prediction system 100, in accordance with some example embodiments. Referring to FIG. 1A, the liver injury prediction system 100 may include a drug analysis engine 110, a data store 120, and a client device 130. As shown in FIG. 1A, the drug analysis engine 110, the data store 120, and the client device 130 may be communicatively coupled via a network 140. The data store 120 may be a database, including, for example, a relational database, a non-structured query language (NoSQL) database, an in-memory database, a graph database, a key-value store, a document store, and/or the like. The client device 130 may be a processor-based device including, for example, a smartphone, a tablet computer, a wearable apparatus, a virtual assistant, an Internet-of-Things (IoT) appliance, and/or the like. The network 140 may be a wired network and/or a wireless network including, for example, a wide area network (WAN), a local area network (LAN), a virtual local area network (VLAN), a public land mobile network (PLMN), the Internet, and/or the like.

In some example embodiments, the drug analysis engine 110 may apply a liver injury prediction model 115 to determine, based at least on one or more features of a drug, the risk of liver injury associated with the drug. For example, in some cases, the liver injury prediction model 115 may determine, based at least on a molecular fingerprint 122, a biological interaction profile 124, and/or one or more molecular properties 126 of a drug, the probability of liver injury associated with the drug. To further illustrate, FIGS. 1B-C depict schematic diagrams illustrating an example of the liver injury prediction model 115. In the example shown in FIGS. 1B-C, the liver injury prediction model 115 may receive from the data store 120, the molecular fingerprint 122, the biological interaction profile 124, and/or the one or more molecular properties 126 of the drug.

In some cases, the liver injury prediction model 115 may be an artificial neural network (ANN), such as a multilayer perceptron (MLP), a convolutional neural network (CNN), and/or the like, having multiple layers of fully or partially connected neurons. For example, in the example shown in FIGS. 1B-C, the liver injury prediction model 115 may include one or more embedding layers 170 that generate a first embedding 175a of the molecular fingerprint 122, a second embedding 175b of the biological interaction profile 124, and a third embedding 175c of the one or more molecular properties 126. Moreover, in some cases, the liver injury prediction model 115 may include one or more prediction layers 185 that determines, based at least on the first embedding 175a of the molecular fingerprint 122, the second embedding 175b of the biological interaction profile 124, and/or the third embedding 175c of the one or more molecular properties 126, a drug-induced liver injury (DILI) risk 180 associated with the drug. For example, in some cases, the first embedding 175a, the second embedding 175b, and the third embedding 175c may be concatenated by fusion into a single vector that is then passed to the one or more prediction layers 185 of the liver injury prediction model 115. In some cases, the one or more prediction layers 185 may include, for example, three (or a different quantity) of prediction layers, that applies an ReLU activation function followed by a softmax function (e.g., in the final prediction layer) in order to assign a classification corresponding to the probability of drug-induced liver injury associated with the drug. Moreover, in some cases, the output of the liver injury prediction model 115 may include a probability distribution across multiple classes (or labels).

Accordingly, in some cases, the liver injury prediction model 115 may be a binary classifier where the drug-induced liver injury risk 180 of the drug determined by the one or more prediction layers 185 includes a first probability of the drug being positive for drug-induced liver injury and a second probability of the drug being negative drug-induced liver injury. Alternatively and/or additionally, the drug-induced liver injury risk 180 of the drug may include a probability of the drug being associated with one or more drug-induced liver injury ranks (e.g., most drug-induced liver injury concern, less drug-induced liver injury concern, no drug-induced liver injury concern, or ambiguous drug-induced liver injury concern).

In some example embodiments, the first embedding 175a, the second embedding 175b, and/or the third embedding 175c may be lower dimensional representations of the corresponding data. For example, while the higher dimensional representation of data may represent the data based on an m-quantity of features (or dimensions), the lower dimensional representation of the same data may represent the data based on a n-quantity of features (or dimensions). Accordingly, as shown in FIG. 1C, the molecular fingerprint 122 of the drug may include 2048 features (or dimensions) whereas the first embedding 175a of the molecular fingerprint 122 may include 256 features (or dimensions) and the biological interaction profile 124 of the drug may include 29959 features (or dimensions) while the second embedding 175b of the biological interaction profile 124 may include 500 features (or dimensions).

It should be appreciated that in some cases, the same one or more embedding layers 170 may generate the first embedding 175a of the molecular fingerprint 122, the second embedding 175b of the biological interaction profile 124, and/or the third embedding 175c of the one or more molecular properties 126. Alternatively, a different embedding layer of the one or more embedding layers 170 may generate each of the first embedding 175a of the molecular fingerprint 122, the second embedding 175b of the biological interaction profile 124, and/or the third embedding 175c of the one or more molecular properties 126.

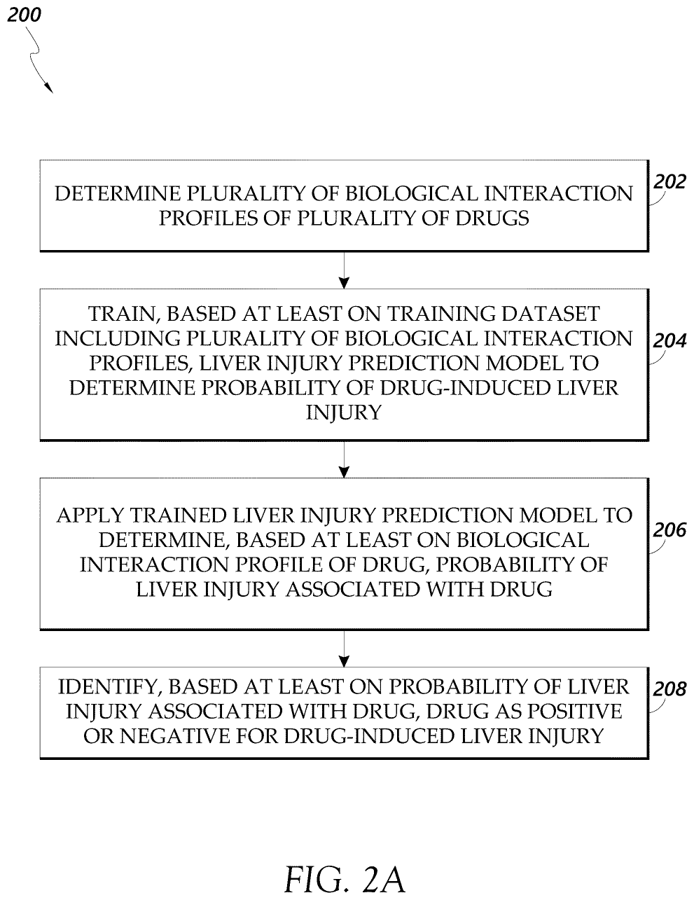

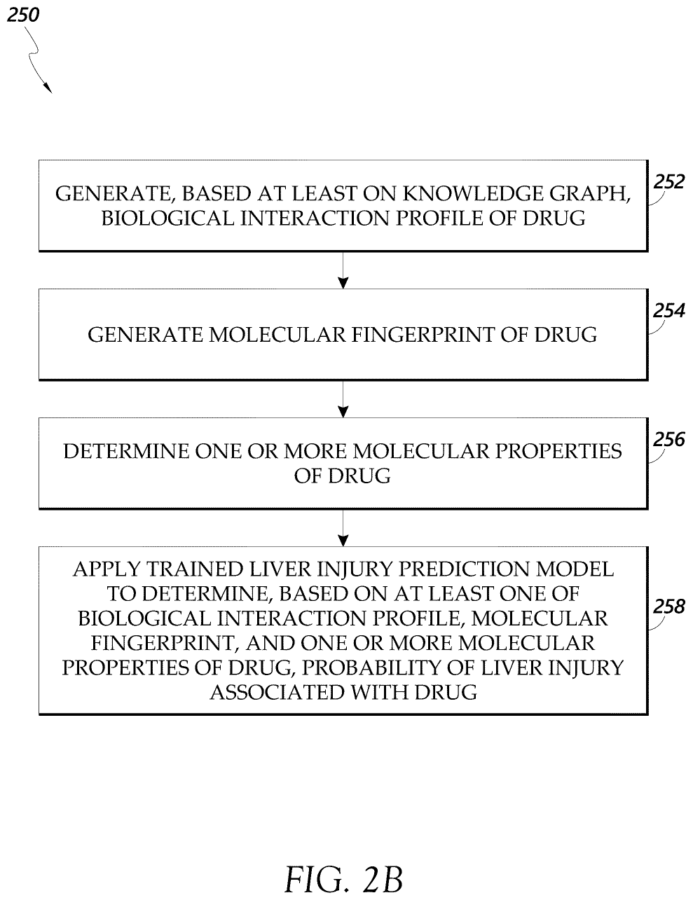

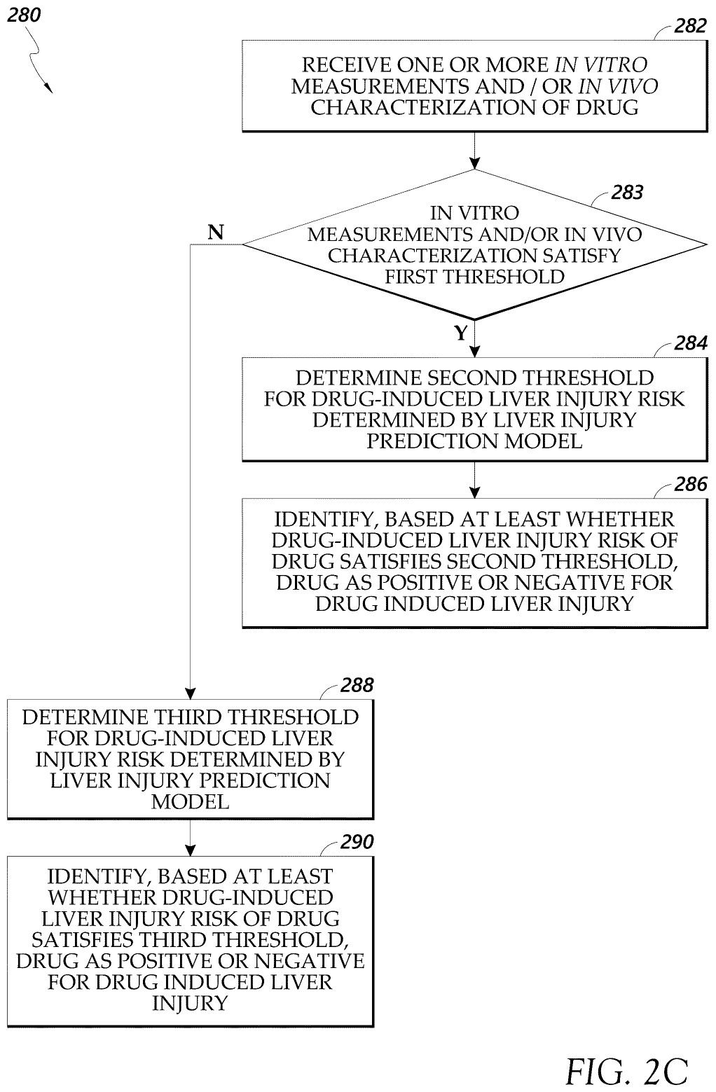

FIG. 2A depicts a flowchart illustrating an example of a process 200 for deep learning-based prediction of drug-induced liver injury, in accordance with some example embodiments. Referring to FIGS. 1A-C and 2A, the process 200 may be performed by the drug analysis engine 110 to train and apply the liver injury prediction model 115 to determine the probability of liver injury associated with a drug (or drug molecule).

At 202, the drug analysis engine 110 may determine a plurality of biological interaction profiles associated with a plurality of drugs. In some example embodiments, the drug analysis engine 110 may determine, for inclusion in a training dataset, a plurality of biological interaction profiles, each of which being representative of one or more effects of a corresponding drug being propagated through one or more protein-protein interactions and biological functions. In some cases, the drug analysis engine 110 may further construct the training dataset to include, for each drug, a molecular fingerprint representative of the molecular structure of the drug. For example, in some instances, the training dataset may include, for each drug, an extended-connectivity fingerprint (ECFP) of the drug. Furthermore, in some cases, the drug analysis engine 110 may construct the training dataset to include, in addition to or instead of the molecular fingerprint of each drug, one or more molecular properties. Examples of molecular properties may include molecular weight, topological surface area, partition coefficient (cLogP), distribution coefficient (cLogD), and/or the like.

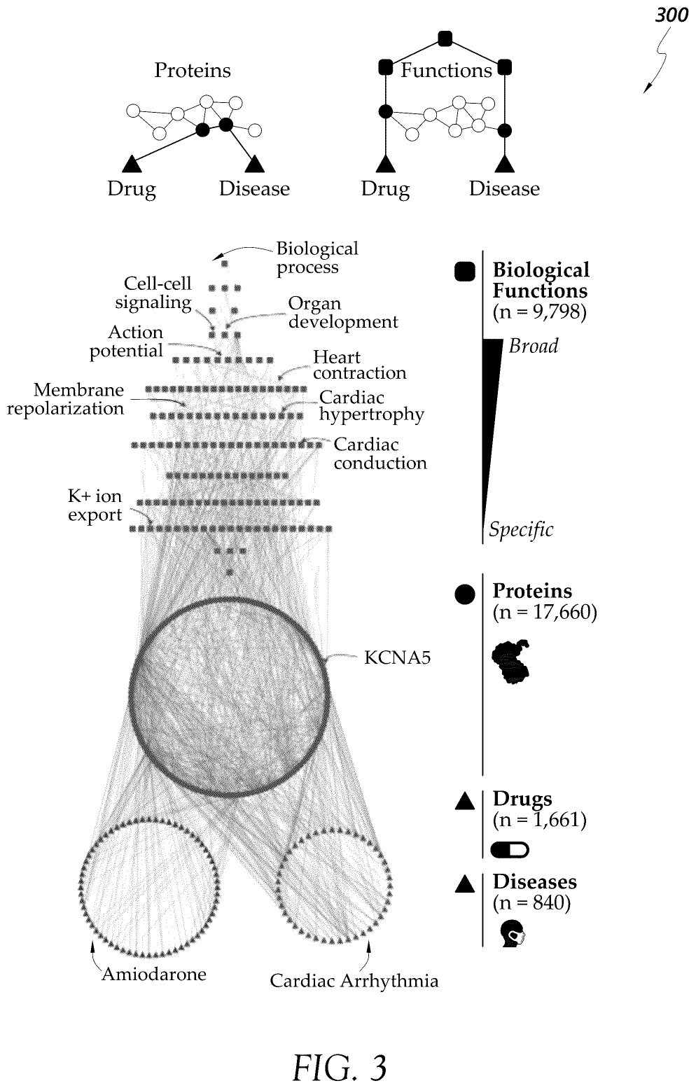

In some example embodiments, the drug analysis engine 110 may determine, based at least on a knowledge graph, each biological interaction profile. In some cases, the knowledge graph may represent the interactions between a variety of different drugs and proteins as well as a hierarchy of biological functions. To further illustrate, FIG. 3 depicts an example of a knowledge graph 300 having a plurality of nodes interconnected by a plurality of edges. In some cases, each node in the knowledge graph 300 may correspond to a drug, a protein, a biological function, or a disease. Meanwhile, each edge in the knowledge graph 300 may correspond to a drug-protein interaction, a disease-protein interaction, a protein-protein interaction, a protein-biological function interaction, or a biological function-biological function interaction between a first node and a second node connected by the edge. For example, in some cases, the knowledge graph 300 may be a network in which 1,661 drugs interact with various target proteins (e.g., as indicated by 8,568 edges interconnecting the corresponding nodes) and 840 diseases interact with the proteins they disrupt through genomic alterations, altered expression, or post-translational modification (e.g., as indicated by 25,212 edges). These protein-level interactions may subsequently propagate through physical interactions with other proteins according to various regulatory, metabolic, kinase-substrate, signaling, and/or binding relationships (e.g., protein-protein interactions between 17,660 proteins as indicated by 387,626 corresponding edges). Alternatively and/or additionally, these proteins may alter biological functions according to a hierarchy of biological functions ranging, for example, from specific processes (e.g., embryonic heart tube elongation) to broader or more general processes (e.g., heart development). For instance the knowledge graph 300 may include a hierarchy of nodes to represent the hierarchical relationship in which

$${}^{‶} negative regulation of response to interferon - {gamma}^{″} {\to }^{is a} {}^{‶} negative regulation of immune {response}^{″} {\to }^{negatively regulates} {}^{‶} immune response {.}^{″}$$

In the context of the knowledge graph 300, the term “biological function” may refer to a process involving molecules (e.g., DNA demethylation), cells (e.g., the mitotic cell cycle), tissues (e.g., muscle atrophy), organ systems (e.g., activation of the innate immune response), and/or the whole organism (e.g., anatomical structure development). Accordingly, an edge in the knowledge graph 300 interconnecting two or more nodes that correspond to different biological relationships may indicate various types of relationship between the biological functions including, for example, regulates, positively regulates, negatively regulates, part of, is a, and/or the like. In the example of the knowledge graph 300 shown in FIG. 3, 34,777 edges interconnect nodes corresponding to proteins and 9,798 biological functions while 22,545 edges interconnect nodes corresponding to different biological functions.

At 204, the drug analysis engine 110 may train, based at least on a training dataset including the plurality of biological interaction profiles, the liver injury prediction model 115 to determine a probability of drug-induced liver injury. In some example embodiments, the drug analysis engine 110 may train the liver injury prediction model 115 based on a training dataset generated to include the biological interaction profile of the plurality of drugs. In some cases, the drug analysis engine 110 may train the liver injury prediction model 115 based on a training dataset generated to include, in addition to the biological interaction profile of the plurality of drugs, the molecular fingerprint and/or one or more molecular properties of each drug. Accordingly, the liver injury prediction model 115 may be trained to determine the probability of drug-induced liver injury based on the biological interaction profile of a drug. In some case, in addition to the biological interaction profile of the drug, the liver injury prediction model 115 may be trained to determine the probability of drug-induced liver injury based on the molecular fingerprint and/or one or more molecular properties of the drug.

At 206, the drug analysis engine 110 may apply the trained liver injury prediction model 115 to determine, based at least on a biological interaction profile of a drug, a probability of liver injury associated with the drug. In some example embodiments, the trained liver injury prediction model 115 may be applied to determine, based on the biological interaction profile of a drug, the probability of the drug causing drug-induced liver injury. In some cases, in addition to the biological interaction profile of the drug, the trained liver injury prediction model 115 may be applied to determine, based at least on the molecular signature and/or one or more molecular properties of the drug, the probability of liver injury associated with the drug. The trained liver injury prediction model 115 may, in some instances, output a first probability of the drug being positive for drug-induced liver injury and a second probability of the drug being negative drug-induced liver injury. Alternatively and/or additionally, the trained liver injury prediction model 115 may output a probability of the drug being associated with one or more drug-induced liver injury ranks (e.g., most drug-induced liver injury concern, less drug-induced liver injury concern, no drug-induced liver injury concern, or ambiguous drug-induced liver injury concern).

At 208, the drug analysis engine 110 may identify, based at least on the probability of liver injury associated with the drug, the drug as positive or negative for drug-induced liver injury. For example, in some cases, the drug analysis engine 110 may determine that the drug is positive for drug-induced liver injury if the probability of the drug being positive for drug-induced liver injury satisfies (or fails to satisfy) one or more thresholds. The drug analysis engine 110 may determine that the drug is negative for drug-induced liver injury if the probability of the drug being negative for drug-induced liver injury satisfies (or fails to satisfy) one or more thresholds. Alternatively, in some cases, the drug analysis engine 110 may determine that the drug is positive for drug-induced liver injury if the probability of the drug being associated with most drug-induced liver injury concern or less drug-induced liver injury concern satisfies (or fails to satisfy) one or more thresholds. Furthermore, the drug analysis engine 110 may determine that the drug is negative for drug-induced injury if the probability of the drug being associated with no drug-induced liver injury concern or ambiguous drug-induced liver injury concern satisfies (or fails to satisfy) one or more thresholds.

In some example embodiments, in addition to the probability of the drug being positive and/or negative for drug-induced liver injury, the drug analysis engine 110 may further identify the drug as being positive or negative for drug-induced liver injury based on one or more in vitro measurements and/or in vivo characterization. For example, in some cases, whether the drug is positive or negative for drug-induced liver injury may be further determined based on one or more in vitro measurements and/or in vivo characterization indicative of the likelihood of the drug causing drug-induced liver injury. Accordingly, in some cases, where the one or more in vitro measurements and/or in vivo characterization associated with the drug satisfy a first threshold, the drug may be identified as positive for drug-induced liver injury if the probability of the drug being positive for drug-induced liver injury (or the probability of the drug being associated with most drug-induced liver injury concern or less drug-induced liver injury concern) satisfies a second threshold. Alternatively, where the one or more in vitro measurements and/or in vivo characterization associated with the drug fails to satisfy the first threshold, the drug analysis engine 110 may identify the drug as being positive for drug-induced liver injury if the probability of the drug being positive for drug-induced liver injury (or the probability of the drug being associated with most drug-induced liver injury concern or less drug-induced liver injury concern) satisfies a third threshold.

FIG. 2B depicts a flowchart illustrating an example of a process 250 for deep learning-based prediction of drug-induced liver injury, in accordance with some example embodiments. Referring to FIGS. 1A-C and 2A-B, the process 250 may be performed by the drug analysis engine 110 to implement operation 206 of the process 200 in which the drug analysis engine 110 applies the trained liver injury prediction model 115 to determine the probability of liver injury associated with a drug (or drug molecule).

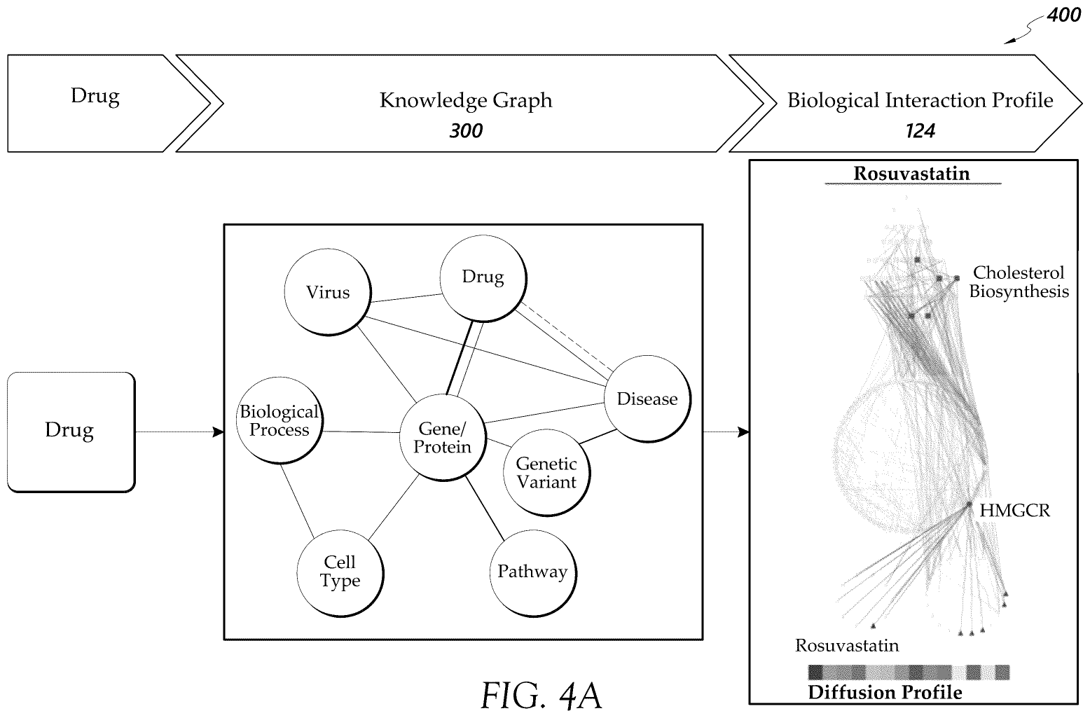

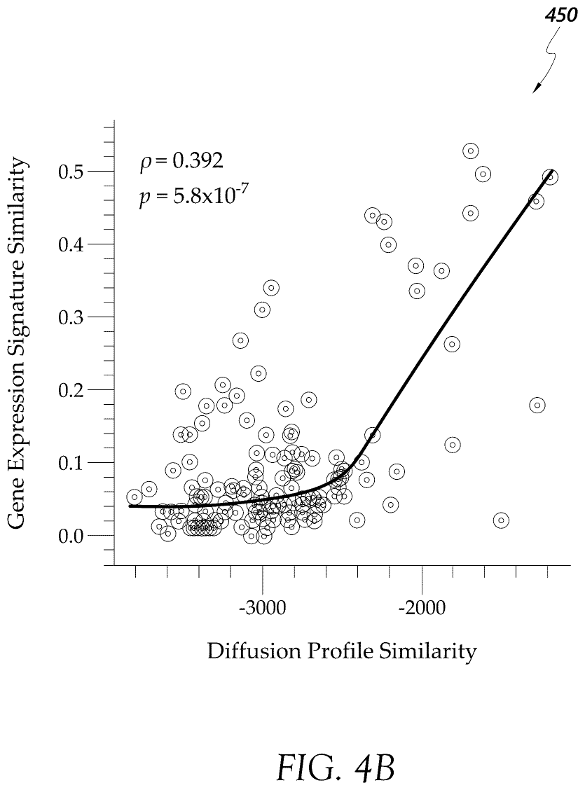

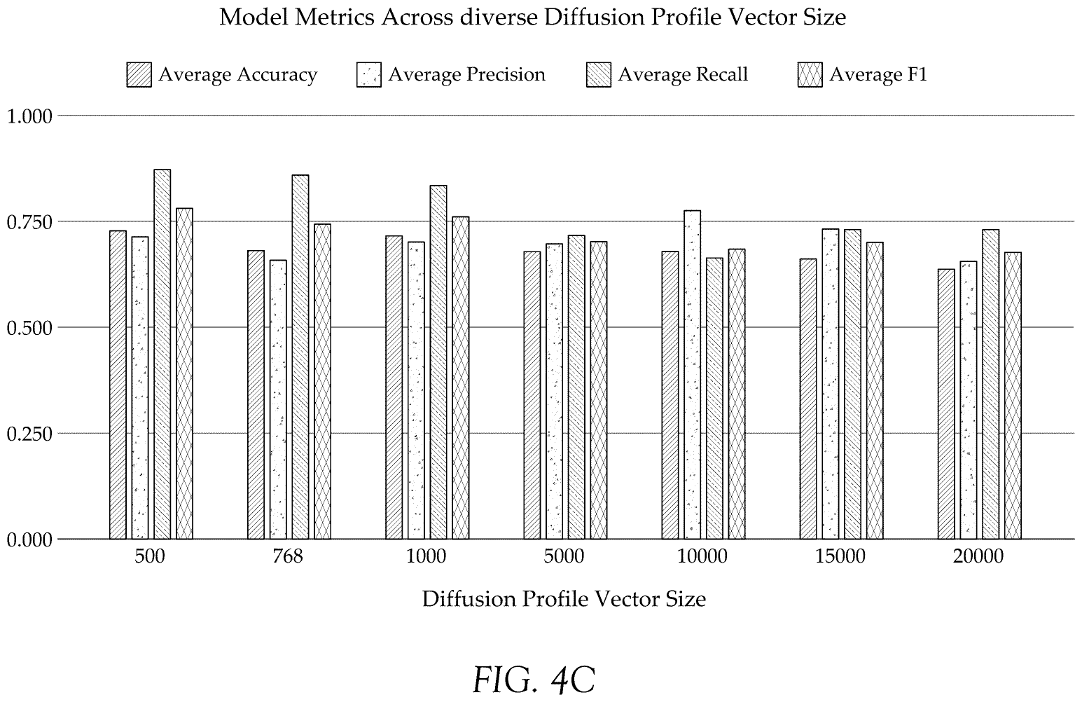

At 252, the drug analysis engine 110 may generate, based at least on a knowledge graph, a biological interaction profile of a drug. For example, FIG. 4A depicts a schematic diagram illustrating an example of a process 400 in which the drug analysis engine 110 generates, based at least on the knowledge graph 300, the biological interaction profile 124 for a drug. As noted, the knowledge graph 300 may include a network of interconnected nodes, each of which corresponding to a drug, a protein, a biological function, or a disease. In some example embodiments, the drug analysis engine 110 may generate the biological interaction profile 124 by performing one or more traversals across the knowledge graph 300. For instance, in some cases, the drug analysis engine 110 may start each traversal of the knowledge graph 300 from a node in the knowledge graph 300 corresponding to the drug. Alternatively, in cases where the drug is a novel drug for which no corresponding node is present in the knowledge graph 300, the one or more traversals across the knowledge graph 300 may start at a node corresponding to a protein affected by the drug. The resulting biological interaction profile 124 may include, for each node in the knowledge graph 300, a frequency of the node being visited during the one or more traversals of the knowledge graph 300.

In some cases, each traversal of the knowledge graph 300 may be a random walk between successive nodes in the knowledge graph 300. In cases where the edges of the knowledge graph 300 are unweighted, the traversal from a first node to a second node in the knowledge graph 300 may include selecting the second node uniformly at random from among the neighboring nodes of the first node. Alternatively, the edges of the knowledge graph 300 may be associated with weights encoding, for example, the relative importance of the corresponding nodes. For example, the weights associated with the edges interconnecting nodes representative of proteins and biological functions may be indicative of how proteins and biological functions at different hierarchical levels have different importance in the effects of drugs and diseases. Where the edges of the knowledge graph 300 are associated with weights, the drug analysis engine 110 may perform one or more biased random walks across the knowledge graph 300. During a biased random walk, the traversal from a first node to a second node in the knowledge graph 300 may include selecting, from the neighboring nodes of the first node, the second node based on the weights associated with the interconnecting edges.

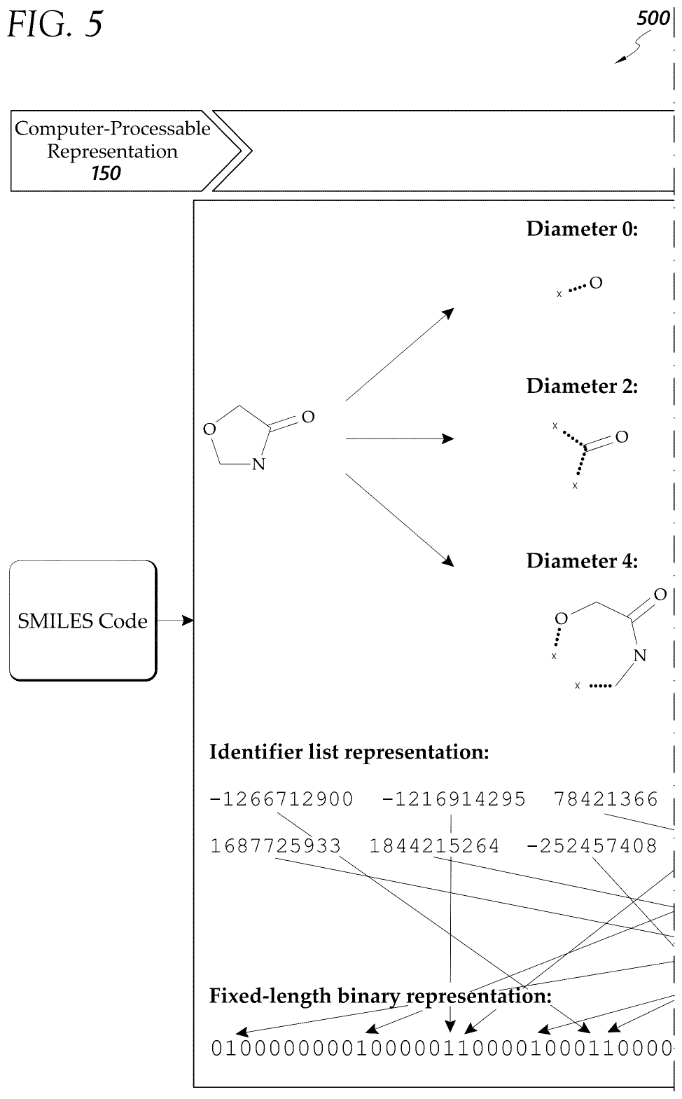

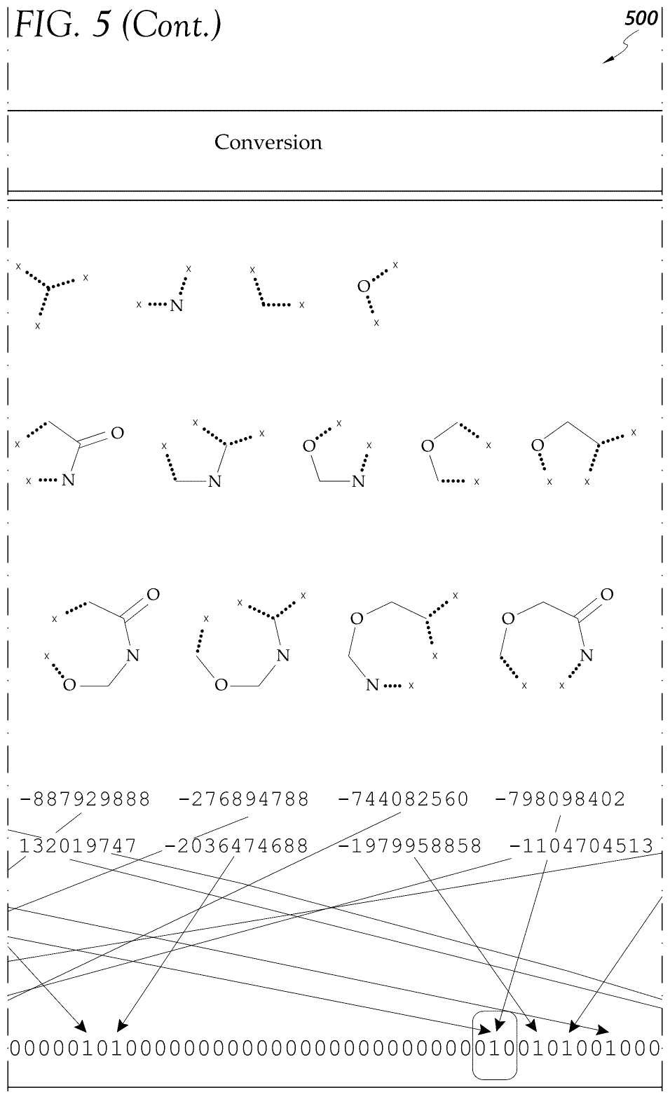

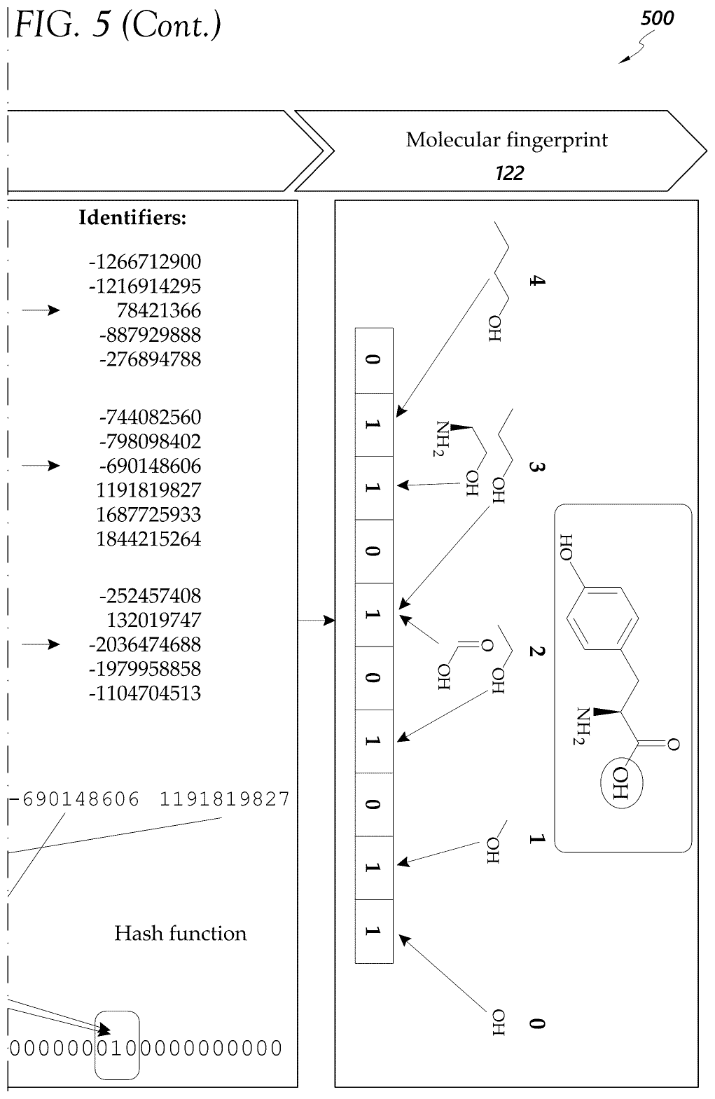

At 254, the drug analysis engine 110 may generate a molecular fingerprint of the drug. FIG. 5 depicts a schematic diagram illustrating an example of a process 500 in which the drug analysis engine 110 generates the molecular fingerprint 122 of the drug. In the example shown in FIG. 5, the molecular fingerprint 122 of the drug may be a circular topological fingerprint such as an extended-connectivity fingerprint (ECFP). Moreover, in some cases, the drug analysis engine 110 may generate the molecular fingerprint 122 of the drug based on a computer-processable representation 150 of the drug's molecular structure such as a Simplified Molecular Input Line Entry System (SMILES) code representation of the drug's molecular structure. For example, in some cases, the drug analysis engine 110 may convert the computer-processable representation 150 of the drug's molecular structure (e.g., isomeric SMILES code of the drug) into a 2048-bit vector D corresponding the extended-connectivity fingerprint (ECFP) of the drug. This fingerprint vector D may be passed through the one or more embedding layers 170 of the liver injury prediction model 115, which outputs the first embedding 175a that is a 256-dimensional hidden representation of the molecular fingerprint 122 of the drug.

At 256, the drug analysis engine 110 may determine one or more molecular properties of the drug. For example, in some cases, the drug analysis engine 110 may determine one or more of the weight, the topological surface area, the partition coefficient (cLogP), and/or distribution coefficient (cLogD) of the drug. Additional details regarding some examples of molecular properties are shown in Table 2 below.

TABLE 2

| Molecular Property | Description | Type |
|---|---|---|
| Molecular Weight | Molecular Weight of | Calculated Property |
| | Compound | |
| Topological Surface Area | Total Polar Surface Area | Calculated Property (surface |
| (TPSA) | | sum over all polar atoms) |
| cLogp | logarithm of a compounds | Calculated Property (measure |
| | partition coefficient between | of hydrophilicity) |
| | n-octanol and water | |
| | log(coctanol/cwater) in its | |
| | neutral form | |
| cLogD7.4 | logarithm of a compounds | Calculated Property (measure |
| | distribution between n- | of lipophilicity and ionization |
| | octanol and water | ability of a compound) |
| | log(coctanol/cwater) at | |
| | various pH | |

At 258, the drug analysis engine 110 may apply the trained liver injury prediction model 115 to determine, based on at least one of the biological interaction profile, the molecular fingerprint, and the one or more molecular properties of the drug, a probability of liver injury associated with the drug. In some example embodiments, the drug analysis engine 110 may apply the trained liver injury prediction model 115 to determine, based on the biological interaction profile 124 of the drug, the probability of liver injury associated with the drug. As shown in FIG. 4B, the liver injury prediction model 115 may determine the probability of liver injury associated with the drug based on the biological interaction profile 124 of the drug instead of drug-induced gene expression data of the drug at least because drugs having similar biological interaction profiles exhibit more similar gene expression signatures. In some cases, the liver injury prediction model 115 may operate on the second embedding 175b of the biological interaction profile 124. The second embedding 175b may be a lower dimensional representation of the biological interaction profile 124 that retains at least some meaningful properties of the biological interaction profile 124 while being more computationally tractable to operate upon than the original high dimensional representation of the biological interaction profile 124. Accordingly, as shown in FIGS. 1B-C, the liver injury prediction model 115 include one or more embedding layers 170 that generates the second embedding 175b of the biological interaction profile 124 before the one or more prediction layers 185 of the liver injury prediction model 115 determines the drug-induced liver injury risk 180 of the drug.

FIG. 4B shows that the performance 450 of the liver injury prediction model 115, for example, as measured based on metrics such as accuracy, precision, recall, and F1 score (e.g., an average value of precision and recall such as harmonic mean and/or the like), may be dependent on the quantity of features (or dimensions) forming the biological interaction profile 124. In some cases, for example, the performance of the liver injury prediction model may be optimal when the second embedding 175b of the biological interaction profile 124 includes 500 features (or dimensions). Moreover, in some cases, in addition to the biological interaction profile 124 of the drug, the liver injury prediction model 115 may determine the probability of liver injury associated with the drug based on at least one of the molecular fingerprint 122 and the one or more molecular properties 126 of the drug. For instance, in the example shown in FIGS. 1B-C, the liver injury prediction model 115 may determine the probability of liver injury associated with the drug based on at least one of the first embedding 175a of the molecular fingerprint 122 and the third embedding 175c of the one or more molecular properties 126.

FIG. 2C depicts a flowchart illustrating an example of a process 280 for deep learning-based prediction of drug-induced liver injury, in accordance with some example embodiments. Referring to FIGS. 1A-C, 2A, and 2C, the process 280 may be performed by the drug analysis engine 110 to implement operation 208 of the process 200 in which the drug analysis engine 110 identifies, based at least on the probability of liver injury associated with the drug, the drug as positive or negative for drug-induced liver injury.

At 282, the drug analysis engine 110 may receive one or more in vitro measurements and/or in vivo characterization of a drug. For example, in some cases, the drug analysis engine 110 may receive one or more in vitro measurements and/or in vivo characterization indicating a quantity of hazard flags associated with the drug.

At 283, the drug analysis engine 110 may determine whether the one or more in vitro measurements and/or in vivo characterization of the drug satisfy a first threshold. For example, in some cases, the drug analysis engine 110 may determine whether the quantity of hazard flags associated with the drug satisfies a first threshold. As will be described in more detail below, the drug analysis engine 110 may impose different thresholds for assessing the drug-induced liver injury risk 180 determined by the liver injury prediction model 115 for the drug based at least on whether the in vitro measurements and/or in vivo characterization of the drug (e.g., the quantity of hazard flags and/or the like) satisfy the first threshold.

At 283-Y, the drug analysis engine 110 may determine that the one or more in vitro measurements and/or in vivo characterization of the drug satisfy the first threshold. Accordingly, at 284, the drug analysis engine 110 may determine a second threshold for a drug-induced liver injury risk determined by the liver injury prediction model 115. Moreover, at 286, the drug analysis engine 110 may identify, based at least on whether the drug-induced liver injury risk of the drug satisfies the second threshold, the drug as positive or negative for drug-induced liver injury. In some example embodiments, the drug analysis engine 110 may determine a second threshold for the drug-induced liver injury risk 180 determined by the liver injury prediction model 115 when the in vitro measurements and/or in vivo characterization of the drug (e.g., the quantity of hazard flags and/or the like) satisfies the first threshold. For example, in some cases, where the drug is associated with fewer than a threshold quantity of hazard flags, the drug analysis engine 110 may determine a higher threshold for the drug-induced liver injury risk 180 than if the drug is associated with more than the threshold quantity of hazard flags. That is, in some cases, where the in vitro measurements and/or in vivo characterization of the drug indicate a lower than threshold likelihood of the drug causing drug induced liver injury, the drug analysis engine 110 may impose a higher threshold for the drug-induced liver injury risk 180 determined by the liver injury prediction model 115 such that the drug is not identified as being positive for drug induced liver injury unless the drug-induced liver injury risk 180 of the drug satisfies the higher threshold.

Alternatively, at 283-N, the drug analysis engine 110 may determine that the one or more in vitro measurements and/or in vivo characterization of the drug fail to satisfy the first threshold. Accordingly, at 288, the drug analysis engine 110 may determine a third threshold for the drug-induced liver injury risk determined by the trained liver injury prediction model 115. Furthermore, at 290, the drug analysis engine 110 may identify, based at least on whether the drug-induced liver injury risk of the drug satisfies the threshold, the drug as positive or negative for drug-induced liver injury. In some example embodiments, the drug analysis engine 110 may determine a third threshold for the drug-induced liver injury risk 180 determined by the liver injury prediction model 115 when the in vitro measurements and/or in vivo characterization of the drug (e.g., the quantity of hazard flags and/or the like) fail to satisfy the first threshold. For example, in some cases, where the drug is associated with more than the threshold quantity of hazard flags, the drug analysis engine 110 may determine a lower threshold for the drug-induced liver injury risk 180 than if the drug is associated with less than the threshold quantity of hazard flags. Accordingly, it should be appreciated that the third threshold may be a different threshold than the second threshold. For instance, in some cases, where the in vitro measurements and/or in vivo characterization of the drug indicate a higher than threshold likelihood of the drug causing drug induced liver injury, the drug analysis engine 110 may impose a lower threshold for the drug-induced liver injury risk 180 determined by the liver injury prediction model 115 such that the drug may be identified as being positive for drug induced liver injury when the drug-induced liver injury risk 180 of the drug satisfies the lower threshold.

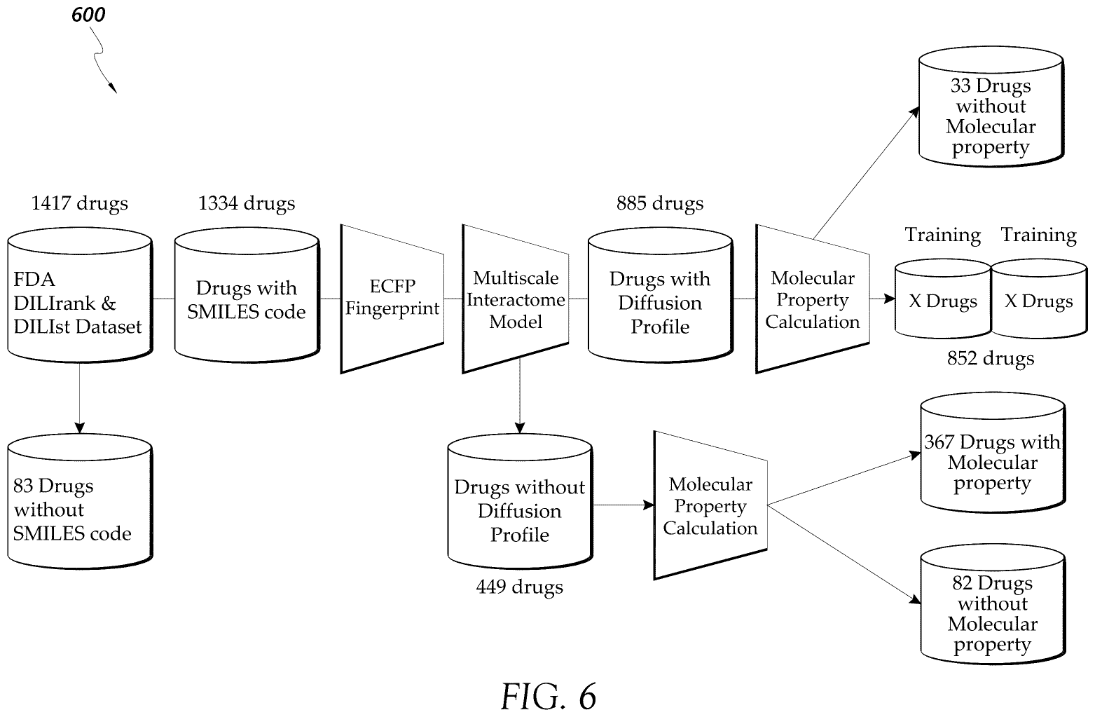

FIG. 6 depicts a schematic diagram illustrating an example of a process 600 for generating training and testing datasets for the liver injury prediction model 115, in accordance with some example embodiments. In some example embodiments, the training and testing datasets for the liver injury prediction model 115 may be generated based on drugs having a known risk of drug-induced liver injury. For example, in some cases, the data associated with drugs having a known risk of drug-induced liver injury may include, from a first data source, 1,036 drugs approved by the Food and Drug Administration (FDA) that have been categorized as being associated with most drug-induced liver injury concern, less drug-induced liver injury concern, no drug-induced liver injury concern, or ambiguous drug-induced liver injury concern. Furthermore, in some cases, the data associated with drugs having a known risk of drug-induced liver injury may include, from a second data source, 1279 drugs, of which 768 are known to be positive for drug-induced liver injury and 511 are known to be negative for drug-induced liver injury. To consolidate data from the two different data sources, drugs that are associated with most drug-induced liver injury concern and less drug-induced liver injury concern may be further categorized as being positive for drug-induced liver injury while drugs that are associated with no drug-induced liver injury concern, or ambiguous drug-induced liver injury concern may be categorized as being negative for drug-induced liver injury. Upon identifying ˜1417 unique drugs from across the two data sources, of which 774 are positive for drug-induced liver injury and 643 are negative for drug-induced liver injury negative, the computer-processable representations (e.g., SMILES code representation and/or the like) of the molecular structure of at least a portion of these drugs (e.g., 1334 drugs in the example shown in FIG. 6) may be retrieved from a third data source. In the example shown in FIG. 6, the resulting training and testing datasets may include the biological interaction profiles, molecular fingerprints, and molecular properties of 852 drugs. In some cases, the drug analysis engine 110 may train and evaluate the liver injury prediction model 115 based on these training and testing datasets.

In some example embodiments, the performance of the liver injury prediction model 115 may be evaluated overall (Task 1) and on a fixed individual dataset (Task 2). For Task 1, data for 852 drugs, including 461 drugs that are positive for drug-induced liver injury and 391 drugs that are negative for drug-induced liver injury, was divided at random into a training dataset (70%), a validation dataset (20%), and a testing dataset (10%) using the shuffle split method. For Task 2, the data associated with the 852 drugs was split into a development set including 716 drugs with balanced data (e.g., 371 drugs that are positive for drug-induced liver injury (51.8%) and 345 drugs that are negative for drug-induced liver injury (48.2%)) and an independent test set including 136 drugs (e.g., 90 drugs that are positive for drug-induced liver injury and 46 drugs that are negative for drug-induced liver injury). The composition of the development set and test set used for Task 2 are shown in Table 3 below.

TABLE 3

Number of compounds

| | | | |
|---|---|---|---|
| | DILI | DILI | |
| Data | Positive | Negatives | Total |

| | | | | | |
|---|---|---|---|---|---|
| Model Development | 371 | (51.8%) | 345 | (48.2%) | 716 |

Dataset

| | | | | | |
|---|---|---|---|---|---|
| Individual Testing | 90 | (66.2%) | 46 | (33.8%) | 136 |

| | | | |
|---|---|---|---|
| Dataset | | | |
| Total | 461 | 391 | 852 |

To evaluate the contribution of features such as biological interaction profiles, molecular fingerprints, and molecular properties to the performance of the liver injury prediction model 115, the liver injury prediction model 115 may be applied to determine the probability of drug-induced liver injury based on different combinations of features. In the first test case, which serves as a baseline, the liver injury prediction model 115 was applied to determine the probability of drug-induced liver injury based on the molecular fingerprint of a drug alone. In the second test case, the liver injury prediction model 115 was applied to determine the probability of drug-induced liver injury based on the molecular fingerprint of the drug along with the drug's biological interaction profile. In the third test case, the liver injury prediction model 115 was applied to determine the probability of drug-induced liver injury based on the molecular fingerprint, the biological interaction profile, and the molecular properties of the drug.

It should be appreciated that the performance of the liver injury prediction model may be evaluated based on metrics including F1, Precision (P), Accuracy (A), and Recall (R). These mathematical expressions of these performance metrics are shown below.

$$F 1 = \frac{2 * Precision * Recall}{Prec i s i o n + R e c a l l}$$

$$Precison = \frac{True Positive}{True Positive + False Positive}$$

$$Recall = \frac{True Positive}{True Positive + False Negative}$$

$$Accuracy = \frac{True Positive + True Negative}{True Positive + True Negative + False Positive + False Negative}$$

As noted, the performance of the liver injury prediction model 115 was evaluated across three different combinations of features. In each of the three aforementioned test cases, the performance of the liver injury prediction model 115 was evaluated by 5-fold cross validation with the shuffle split method. Furthermore, as noted, Task 1 includes evaluating the overall performance of the liver injury prediction model 115 while Task 2 includes evaluating the performance of the liver injury prediction model 115 on a fixed individual dataset.

Task 1 was performed using the dataset including 852 molecules (e.g., 461 drugs that are positive for drug-induced liver and 391 drugs that are negative for drug-induced liver injury) that was divided at random into a training dataset (70%), a validation dataset (20%), and a testing dataset (10%) using the shuffle split method. The evaluation of the performance of the liver injury prediction model may be performed based on a five-fold cross validation with 20 epochs in which the performance of the liver injury prediction model 115 was assessed for the three aforementioned test cases, each which having a different combination of features (e.g., molecular fingerprint alone, molecular fingerprint with biological interaction profile, and molecular fingerprint with biological interaction profile and molecular properties). For each test case, the performance of the liver injury prediction model 115 was evaluated based on the metrics F1, Recall, Precision, and Accuracy. As the results in Table 4 indicate, the second test case in which the liver injury prediction model 115 operated on molecular fingerprints combined with biological interaction profile achieved the best precision at 0.718 while the third test case in which the liver injury prediction model 115 operated on a combination of molecular fingerprints, biological interaction profiles, and molecular properties achieved the best performance across F1, Recall, and Accuracy (e.g., 0.792, 0.898, and 0.753 respectively). The results shown in Table 4 further indicate that liver injury prediction model 115 is able to achieve performance comparable to that of in silico techniques but without any reliance on drug-induced gene expression data.

TABLE 4

| | Hyperparameters | F1 | Recall | Precision | Accuracy |
|---|---|---|---|---|---|
| Model 1: ECFP | Learning Rate = | 0.754 | 0.876 | 0.666 | 0.702 |
| | 0.0005 | (0.036) | (0.113) | (0.013) | (0.025) |
| | Weight Decay = | | | | |
| | 0.000008 | | | | |
| Model 2: ECFP + | Learning Rate = | 0.779 | 0.853 | 0.718 | 0.747 |
| Biological interaction | 0.00055 | (0.021) | (0.040) | (0.030) | (0.025) |
| profile | Weight Decay = | | | | |
| | 0.000012 | | | | |
| Model 3: ECFP + | Learning Rate = | 0.792 | 0.898 | 0.711 | 0.753 |
| Biological interaction | 0.0045 | (0.008) | (0.060) | (0.028) | (0.010) |
| profile + Molecular | Weight Decay = | | | | |
| Properties | 0.000008 | | | | |

For Task 2, the performance of the liver injury prediction model 115 is validated based on a fixed individual dataset in which 716 compounds were selected for training the liver injury prediction model 115 using the same strategy as Task 1 while the remaining 136 compounds (e.g., 90 drugs that are positive for drug-induced liver injury and 46 drugs that are negative for drug-induced liver injury) used for performance evaluation is selected separately from the training set. As the results in Table 5 show, the liver injury prediction model 115 was able to achieve 0.804 in precision and 0.797 in F1 while providing consistent predictive power on the individual test dataset. With a soft voting system from the 5-fold cross validation models, the probability of positive prediction are weight-averaged to obtain the risk of drug-induced liver injury. When 0.5 is used as the threshold, the performance the liver injury prediction model 115 in the third test case that uses the biological interaction profiles, molecular fingerprints, and the molecular properties of drugs increased 1.5% for F1 score and 4% for recall.

TABLE 5

| External validation | Hyperparameters | F1 | Recall | Precision | Accuracy |
|---|---|---|---|---|---|
| Model 1: ECFP | Learning Rate = | 0.766 | 0.753 | 0.784 | 0.699 |
| | 0.00045 | (0.042) | (0.078) | (0.026) | (0.041) |
| | Weight Decay = | | | | |
| | 0.000009 | | | | |
| Model 2: ECFP + | Learning Rate = | 0.784 | 0.773 | 0.797 | 0.719 |
| Biological | 0.0005 | (0.014) | (0.037) | (0.015) | (0.011) |
| interaction profile | Weight Decay = | | | | |
| | 0.00001 | | | | |
| Model 3: ECFP + | Learning Rate = | 0.797 | 0.791 | 0.804 | 0.734 |
| Biological | 0.0045 | (0.0229) | (0.056) | (0.018) | (0.021) |
| interaction profile + | Weight Decay = | | | | |
| Molecular | 0.000012 | | | | |
| Properties | | | | | |

In some example embodiments, the performance of the liver injury prediction model 115 may be further enhanced through hyperparameter tuning. For example, in some cases, a grid search of the hyperparameter space may be conducted to identify hyperparameters that maximize the performance of the liver injury prediction model 115. Some examples of optimal hyperparameters, including the number of epochs, learning rate, and weight decay, for Task 1 and Task 2 are shown in Tables 4 and 5, respectively.

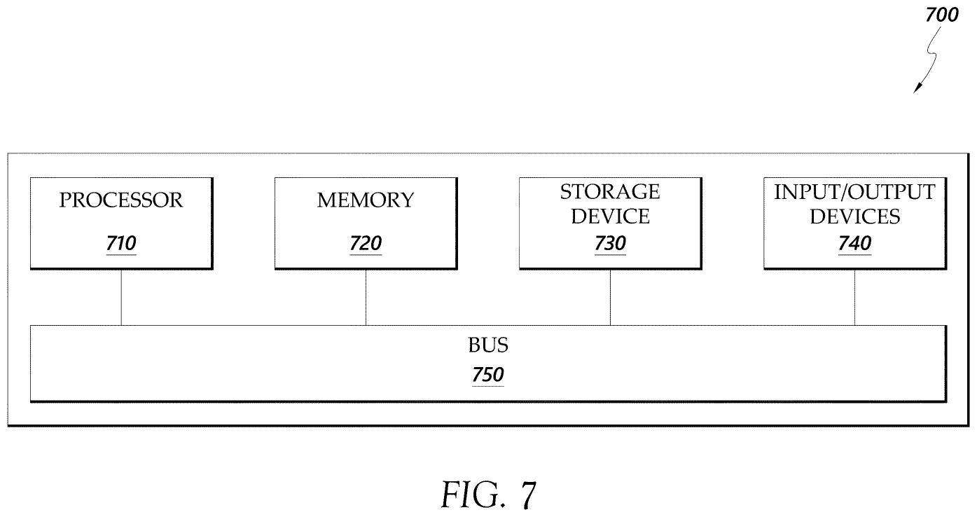

FIG. 7 depicts a block diagram illustrating an example of a computing system 700 consistent with implementations of the current subject matter. Referring to FIGS. 1-7, the computing system 700 can be used to implement the drug analysis engine 110 and/or any components therein.

As shown in FIG. 7, the computing system 700 can include a processor 710, a memory 720, a storage device 730, and an input/output device 740. The processor 710, the memory 720, the storage device 730, and the input/output device 740 can be interconnected via a system bus 750. The processor 710 is capable of processing instructions for execution within the computing system 700. Such executed instructions can implement one or more components of, for example, the drug analysis engine 110. In some example embodiments, the processor 710 can be a single-threaded processor. Alternately, the processor 710 can be a multi-threaded processor. The processor 710 is capable of processing instructions stored in the memory 720 and/or on the storage device 730 to display graphical information for a user interface 135 provided via the input/output device 740.

The memory 720 is a computer readable medium such as volatile or non-volatile that stores information within the computing system 700. The memory 720 can store data structures representing configuration object databases, for example. The storage device 730 is capable of providing persistent storage for the computing system 700. The storage device 730 can be a solid-state drive, a floppy disk device, a hard disk device, an optical disk device, or a tape device, or other suitable persistent storage means. The input/output device 740 provides input/output operations for the computing system 700. In some example embodiments, the input/output device 740 includes a keyboard and/or pointing device. In various implementations, the input/output device 740 includes a display unit for displaying graphical user interfaces 135.

According to some example embodiments, the input/output device 740 can provide input/output operations for a network device. For example, the input/output device 740 can include Ethernet ports or other networking ports to communicate with one or more wired and/or wireless networks (e.g., a local area network (LAN), a wide area network (WAN), the Internet).

In some example embodiments, the computing system 700 can be used to execute various interactive computer software applications that can be used for organization, analysis and/or storage of data in various formats. Alternatively, the computing system 700 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface 135 provided via the input/output device 740. The user interface 135 can be generated and presented to a user by the computing system 700 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random query memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, recurrent provided to the user can be any form of sensory recurrent, such as for example visual recurrent, auditory recurrent, or tactile recurrent; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

### APPENDIX A

The following experimental results correspond to embodiments described herein for use of multimodal learning in drug development and drug-induced liver injury risk prediction.

For drug development, diverse experiments can be used to understand properties of drug candidates and guarantee drug safety for humans. Each data modality can not only provide insight for molecules but can also represent valuable characteristics to enable computational analysis. Multimodal learning was proposed to build a model that can learn information from multiple modalities for insight discovery.

During drug development, drug-induced liver injury (DILI) is one of many serious concerns for patient safety and over the last 50 years has been the major cause of drug candidate attrition and market withdrawal. In extreme cases, DILI results in liver transplant or death. Therefore, predicting risk of liver injury early in the drug development process can not only lower risk for severe hepatic outcome for patients but can also save hundreds of millions of dollars and years of research efforts. However, DILI can closely resemble known liver diseases, often attributable to two types, intrinsic and idiosyncratic. Intrinsic DILI can refer to predictable and dose-dependent liver injury. Idiosyncratic DILI can be less dependent on dose, route or duration of administration, but can be associated with host factors and individual susceptibility like gene variants and demographics. Due to multiple etiologies and multifactorial mechanisms, several factors other than the generation of toxic intermediate(s) from parental drug metabolism, such as, physiological and genetic factors as well as altered immunological responses were studied to be relevant to DILI. Therefore, DILI risk prediction is a challenging task.

Over the last several decades, many in vivo and in vitro approaches have been developed to evaluate the risk of DILI. However, such studies are expensive, time-consuming, and may not reliably predict DILI in patients. Thus, novel techniques and methods for evaluating DILI risk are proposed and described herein.

Several DILI predicted models in Table 6 were proposed to predict the risk of drug toxicity. From this table, we observe that two main limitations were encountered in evaluating and comparing their performance. The first challenge involved the inconsistent DILI categorization scheme used across different reported DILI classifications. The second challenge dealt with the data availability for feature engineering and model building. For example, drug induced gene expression or assay data may be unavailable to all drugs, which highly limits the sample size for model development and evaluation. To conquer the first challenge, a comprehensive clinical DILI annotation can be created by combining the most reliable DILI annotation datasets (DILIRank and DILIst) to increase sample size. For the second challenge, we explored another key feature which not only utilizes biomedical knowledge graphs with prior knowledge of biological functions and protein interactions but also calculates the importance of nodes within the network. The feature is called a “diffusion profile”. A random walk algorithm can be used to compute a diffusion profile where it starts at a random drug node. At every step, the random walker can restart its walk or jump to an adjacent node based on optimized weights. After many walks, the diffusion profile can measure how often every node was visited and thereby can capture its effect on every protein and biological function. With the calculated diffusion profile, it can capture important biological function & protein-protein interaction of a drug. The statistics analysis also validated that drugs with more similar diffusion profiles have more similar gene expression signatures. In this way, all drugs were described by ECFP fingerprints and molecular properties calculated based on SMILE code and drug diffusion profile reflecting biological functions and physical protein-protein interactions from biomedical knowledge graphs. The predictive model can be trained through a multi-layer perceptron (MLP) neural network and evaluated using an external dataset.

TABLE 6

| | | | | Performance |
|---|---|---|---|---|
| Method | Features | Model | DILI Annotated data | (accuracy) |
| Graph Neural Network | Molecular | Graph | Train/Test with 483 | ACC = 0.773 |
| for Drug-induced Liver | Fingerprints + | Neural | drugs from LTKB | F-1 = 0.7883 |
| Injury Prediction using | Gene | Network | Most-DILI-Concern: 114 | |
| Transcriptome Data | Expression + | | Less-DILI-Concern: 268 | |
| (GLIT) (2020) | Pathway + | | No-DILI-Concern: 101 | |
| | Dose | | | |
| Predictive Model for | Molecular | Deep | Train/Test with from | ACC = 0.731 |
| Drug-Induced Liver | Fingerprints | Neural | DILIrank + LTKB | Sensitivity = |
| Injury Using Deep | | Network | DILI Positive: 461 | 0.714 |
| Neural Networks | | (DNN) | DILI Negative: 479 | Specificity = |
| Based on | | model | | 0.750 |
| Substructure Space | | | | |
| (2021) | | | | |
| Integration of human | Gene | Random | From LTKB | AUC = 0.73 |
| cell lines gene | Expression + | Forest | DILI Concern: 179 | |
| expression and | Molecular | | No-DILI Concern: 54 | |
| chemical properties of | Descriptors | | | |
| drugs for Drug | | | | |
| Induced Liver Injury | | | | |
| prediction (2021) | | | | |
| DeepDILI: Deep | Molecular | Combined | Train/Test with 1002 | ACC = 0.687 |
| learning-powered | Descriptors | machine | drugs from DILIst | Recall = 0.805 |
| drug-induced liver | | learning | DILI Positive: 604 | F-1 = 0.755 |
| injury prediction using | | (ML) | DILI Negative: 398 | |
| model-level | | algorithms | | |
| representation (2020) | | with a deep | | |
| | | learning | | |
| | | framework | | |
| DILI-Stk: An | Molecular | Ensemble | 2170 compounds from | ACC = 0.77 |
| Ensemble Model for | Descriptors | Model (12 | FDA, Orange Book & | Specificity = 0.72 |
| the Prediction of | | different | China National | F-1 = 0.79 |
| Drug-induced Liver | | machine | Knowledge | |
| Injury of Drug | | learning | Infrastructure database | |
| Candidates (2022) | | models) | 1146 DILI-positive | |
| | | | 1024 DILI-negative | |

FIG. 1C provides an overview of the proposed model architecture. The details of data preparation, feature generation, and model development will be described in the following subsections. The DILI data was annotated based on the following methods. The DILI annotation data for this study can be collected from both datasets, including DILIRank and DILIst. DILIrank includes 1,036 FDA-approved drugs that are divided into four classes (vMost-, vLess-, vNo-DILI concern, and Ambiguous-DILI-concern) according to their potential for causing DILI. In addition to DILIRank, DILIst contains information for 1279 drugs, of which 768 are DILI positive and 511 are DILI negatives. To consolidate two datasets, all drugs were mapped using Drugbank Identifiers (DBIDs). For DILI annotation, 1,036 drugs from DILIRank are binary categorized into ‘positives’ if drugs have vMost- and vLess-DILI concern and ‘negatives’ if drugs have vNo- and Ambiguous-DILI-concern. After duplicate removal and class assignment, the final dataset consisted of ˜1417 unique drugs, of which 774 are DILI positives and 643 are DILI negatives.

Feature generation can be performed via the following methods. To utilize molecular descriptors as DILI prediction features, Extended Connectivity Fingerprint (ECFP), which is a type of molecular fingerprint explicitly designed to capture molecular features relevant to molecular activity, can be employed for generating the structure feature of a drug. In this task, isomeric SMILE codes for drugs acquired from PubChem can be converted to 2048-bit ECFP fingerprint vector D generated by the Morgan Algorithm from the rdkit library. To represent the fingerprint vector D most efficiently, a 2048-bit vector D can be fed to a multi-layer perceptron (MLP) to obtain the 256-dimensional hidden representation (drug embedding) from the last layer of a neural network with three layers. To deploy more potential features for DILI prediction, 4 additional molecular properties in Table 7 can be taken into consideration for model development.

TABLE 7

| Molecular Property | Description | Type |
|---|---|---|
| Molecular Weight | Molecular Weight of | Calculated Property |
| | Compound | |
| Topological Surface Area | Total Polar Surface Area | Calculated Property (surface |
| (TPSA) | | sum over all polar atoms) |
| cLogp | logarithm of a compounds | Calculated Property |
| | partition coefficient between | (measure of hydrophilicity) |
| | n-octanol and water | |
| | log(coctanol/cwater) in its | |
| | neutral form | |
| cLogD7.4 | logarithm of a compounds | Calculated Property |
| | distribution between n- | (measure of lipophilicity and |
| | octanol and water | ionization ability of a |
| | log(coctanol/cwater) at | compound) |
| | various pH | |

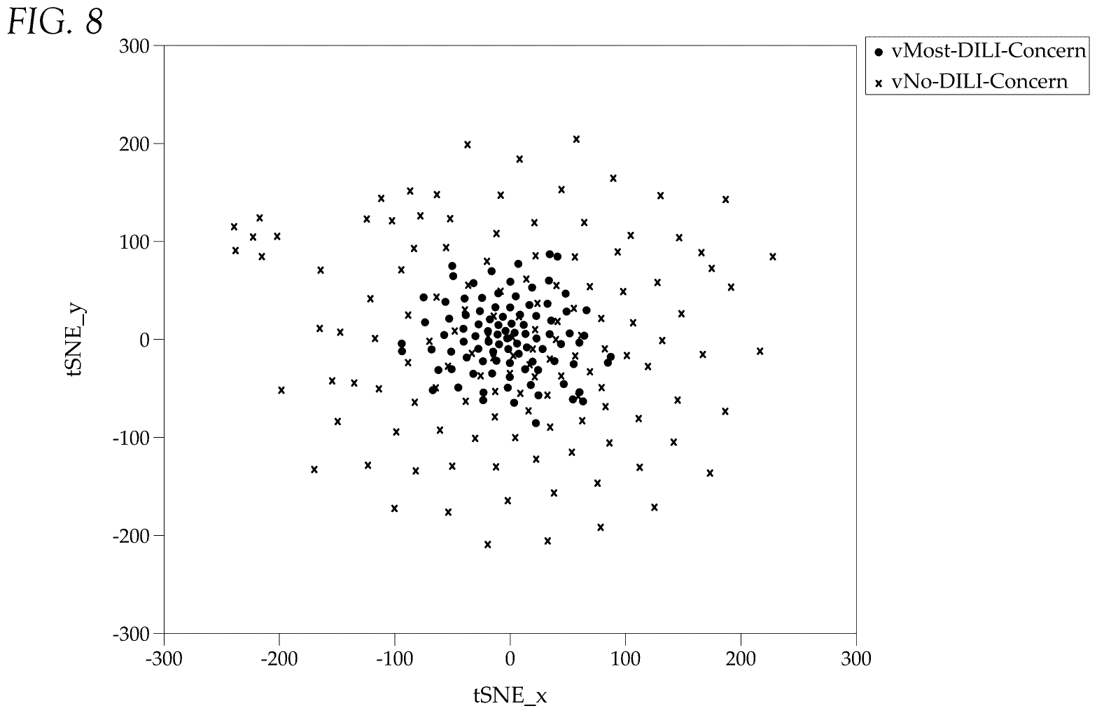

By using the diffusion profile as the feature, various dimensions of embedding can be evaluated according to the model performance. The size of 500-dimensional embeddings can be determined to achieve optimal performance shown in FIG. 4C. Then, a 29959-bit vector is fed to a multi-layer perceptron (MLP) to obtain the 500-dimensional hidden representation from the last layer of a neural network with three layers. In FIG. 8, diffusion profile embedding (500-bit vector) can be converted to vector space using the tSNE plot. We identified that the Most-DILI and No-DILI may be not perfectly separated by this feature. However, it already provides a substantial signal for DILI prediction.

The final dataset includes 1417 FDA-approved drugs, of which 774 are DILI positives and 643 are DILI negatives. Among those drugs, 1334 drugs' Smile Codes can be retrieved from the PubChem and Chemical Entities of Biological Interest (ChEBI) database. Regarding the availability of all features (diffusion profiles, molecular descriptors/properties), finally, 852 drugs (See FIG. 6) can be obtained for model development.

To evaluate the overall performance of the predictive model (Task 1) and the performance of the model on a fixed individual dataset (Task 2), two tasks can be performed. For task 1, 852 compounds (461 DILI Positive, 391 DILI Negative) can be randomly divided into training (70%), validation (20%), and testing sets (10%) using the shuffle split method. For Task 2, the dataset can be split into 716 drugs with balanced data (371 DILI Positive [51.8%] and 345 DILI Negative [48.2%]) and 136 drugs (90 DILI positives and 46 DILI negatives) for the development set and the independent test set, respectively (Table 8).

TABLE 8

Number of compounds

| | | | |
|---|---|---|---|
| | DILI | DILI | |
| Data | Positive | Negatives | Total |

| | | | | | |
|---|---|---|---|---|---|
| Model Development | 371 | (51.8%) | 345 | (48.2%) | 716 |

Dataset

| | | | | | |
|---|---|---|---|---|---|
| Individual Testing | 90 | (66.2%) | 46 | (33.8%) | 136 |

| | | | |
|---|---|---|---|
| Dataset | | | |
| Total | 461 | 391 | 852 |

Shown in FIG. 1C, we deployed multi-layer perceptrons (MLPs) to transform embedding input vectors and obtain hidden representations from three different features (molecular descriptor, molecular property, and diffusion profile from a multiscale interactome model). Specifically, the molecular descriptor can be transferred from a 2048-bit vector to a 256-dimensional hidden representation. For the diffusion profile of drugs, the size of embeddings can be fine-tuned from 29959 to 500 according to the model performance (FIG. 4C). Finally, the early fusion strategy can be applied to concatenate embeddings for molecular descriptors and diffusion profiles along with the four molecular properties into a single vector. The vector can then be fed to the three layers of the MLP model to predict binary classification probability (via softmax) with the ReLU activation function.

To evaluate the contribution of features to the predictive models, three predictive models with three different combinations of features can be conducted. Model 1 utilizing only ECFP molecular descriptors can be built as a baseline model. Model 2 can include additional feature diffusion profiles to understand the contribution of the Diffusion profile while Model 3 can use all features in the predictive model.

In this study, three evaluation metrics including F1, Precision (P), Accuracy (A), and Recall (R) can be used to evaluate the model performance. These mathematical expressions of these evaluation metrics are specified below:

$$F 1 = \frac{2 * Precision * Recall}{Prec i s i o n + R e c a l l}$$

$$Precison = \frac{True Positive}{True Positive + False Positive}$$

$$Recall = \frac{True Positive}{True Positive + False Negative}$$

$$Accuracy = \frac{True Positive + True Negative}{True Positive + True Negative + False Positive + False Negative}$$

Three models with different combinations of features can be used to predict the risk of DILI for FDA-approved drugs. Their performance was evaluated by 5-fold cross validation with the shuffle split method. To evaluate the overall performance of the predictive model (Task 1) and the performance of the model on a fixed individual dataset (Task 2), two tasks can be performed.

For task 1, we used the dataset of 852 compounds (461 DILI Positive, 391 DILI Negative) which can be randomly divided into training (70%), validation (20%), and testing sets (10%) using the shuffle split method. Model evaluation can be based on the fivefold cross validation with 20 epochs where each model was evaluated with different feature combinations (ECFP, ECFP+Diffusion Profile, ECFP+Diffusion Profile+Molecular Properties). For each model, we assessed the performance using F1, Recall, Precision, Accuracy and provided results in table x, the model 2 with ECFP+Diffusion Profile achieved the best precision with 0.718 and model 3 achieved best performance for F1, Recall, Accuracy of 0.792, 0.898, 0.753. The model performance shown in table 3 (Table 9) can be very competitive with other DILI prediction models shown in Table 6.

TABLE 9

| | Hyperparameters | F1 | Recall | Precision | Accuracy |
|---|---|---|---|---|---|
| Model 1: ECFP | Learning Rate = 0.0005 | 0.754 | 0.876 | 0.666 | 0.702 |
| | Weight Decay = 0.000008 | (0.036) | (0.113) | (0.013) | (0.025) |
| Model 2: ECFP + | Learning Rate = 0.00055 | 0.779 | 0.853 | 0.718 | 0.747 |
| Diffusion Profile | Weight Decay = 0.000012 | (0.021) | (0.040) | (0.030) | (0.025) |
| Model 3: ECFP + | Learning Rate = 0.0045 | 0.792 | 0.898 | 0.711 | 0.753 |
| Diffusion Profile + | Weight Decay = 0.000008 | (0.008) | (0.060) | (0.028) | (0.010) |
| Molecular Properties | | | | | |

As shown in Table 9, a performance comparison based on different feature combinations results (Task 1: model building with 5 folds cross-validation with 20 epochs, train: 596 (70%), validation: 170 (20%), test: 86 (10%) compounds) is provided.

For Task 2, to validate performance on a fixed individual dataset, 716 compounds can be selected for model development using the same strategy of Task 1 and the rest of 136 compounds (90 DILI positive, 46 DILI negative) entirely separated from the training set can be used for performance evaluation. From Table 10, we can observe that the model predicts well with 0.804 in precision, 0.797 in F1 and provides consistent predictive power on the individual test dataset. With a soft voting system from the 5-fold CV models, the probability of positive prediction can be weight-averaged to obtain the final score. Using the 0.5 as the threshold, the performance on model 3 increases with 1.5% on F1 score & 4% on Recall.

TABLE 10

| External validation | Hyperparameters | F1 | Recall | Precision | Accuracy |
|---|---|---|---|---|---|
| Model 1: ECFP | Learning Rate = 0.00045 | 0.766 | 0.753 | 0.784 | 0.699 |
| | Weight Decay = 0.000009 | (0.042) | (0.078) | (0.026) | (0.041) |
| Model 2: ECFP + | Learning Rate = 0.0005 | 0.784 | 0.773 | 0.797 | 0.719 |
| Diffusion Profile | Weight Decay = 0.00001 | (0.014) | (0.037) | (0.015) | (0.011) |
| Model 3: ECFP + | Learning Rate = 0.0045 | 0.797 | 0.791 | 0.804 | 0.734 |
| Diffusion Profile + | Weight Decay = 0.000012 | (0.0229) | (0.056) | (0.018) | (0.021) |
| Molecular Properties | | | | | |

As shown in Table 10, a performance comparison based on different feature combinations results (Task 2: model building with 5 folds cross-validation with 20 epochs, train: 716 compounds and fixed external validation set of 136 compounds: 90 DILI-positives, 46 DILI-negatives) can be provided. For Task 2, to validate performance on a fixed individual dataset, 716 compounds were selected for model development using the same strategy of Task 1 and the rest of 136 compounds (90 DILI positive, 46 DILI negative) entirely separated from the training set can be used for performance evaluation.

To optimize the model, hyperparameters were searched to maximize performance on validation sets by a grid search. The optimal hyperparameters of the proposed model can include the number of epochs, learning rate, and weight decay were shown in Table 9 and Table 10 for Task 1 and Task 2, respectively. For the Model 3 in Task 1, a learning rate of 0.0045, and a weight decay of 0.000012 can be obtained to achieve optimized performance based on F1 while a learning rate of 0.0045, and a weight decay of 0.000008 can be chosen for the Model 3 in Task 2.

In addition to FDA-approved drugs, we tested our model on a study molecule “Tolebrutinib”, a BTK inhibitor. This drug is used to treat multiple sclerosis (MS) and myasthenia gravis, however, the majority of cases of drug-induced liver injury have been reported in participants with medical histories that may predispose them to this complication Due to this, FDA placed a clinical hold on phase III studies of Tolebrutinib. To predict DILI for Tolebrutinib, the SMILE code (https://pubchem.ncbi.nlm.nih.gov/compound/Tolebrutinib) was obtained from PubChen. The ECFP fingerprint and molecule properties (Molecular Weight: 455.51, Topological Surface Area (TPSA): 95; cLogp: 3.9; cLogD7.4: 3.9) of Tolebrutinib were calculated using the DILI prediction model and methods described in embodiments herein. The diffusion profile of Tolebrutinib was generated from the knowledge graph with an additional drug node (Tolebrutinib) linking to BIK gene. Then, with our DILI prediction model, Tolebrutinib was classified as DILI-positive with a score of 0.52445 using only its chemical structure and target information. The result shows that our model can be used for screening the study molecules in the pre-clinical drug development stage for potential DILI risk assessment.

What is claimed is:

1. A computer-implemented method, comprising:
determining, based at least on a knowledge graph, a plurality of biological interaction profiles associated with a plurality of drugs, the knowledge graph being representative of a plurality of interactions between the plurality of drugs, a plurality of proteins, and a hierarchy of biological functions, and each biological interaction profile of the plurality of biological interaction profiles representative of one or more effects of a corresponding drug being propagated through one or more protein-protein interactions and biological functions;
training, based at least on a training dataset including the plurality of biological interaction profiles, a liver injury prediction model to determine a probability of drug induced liver injury; and
applying the trained liver injury prediction model to determine, based at least on a biological interaction profile of a drug, a probability of liver injury associated with the drug.

2. The method of claim 1, wherein the knowledge graph includes a plurality of nodes interconnected by a plurality of edges, wherein each node of the plurality of nodes is representative of a drug, a protein, a biological function, or a disease, and wherein each edge of the plurality of edges is representative of a drug-protein interaction, a disease-protein interaction, a protein-protein interaction, a protein-biological function interaction, or a biological function-biological function interaction between a first node and a second node connected by the edge.

3. The method of claim 2, further comprising:
determining each biological interaction profile of the plurality of biological interaction profiles by performing one or more random walks between a first node associated with the corresponding drug and a second node associated with a disease, wherein each biological interaction profile of the plurality of biological interaction profiles includes, for each node of the plurality of nodes, a frequency of the node being visited during the one or more random walks.

4. The method of claim 1, further comprising:
training, based at least on the training dataset, the liver injury prediction model to generate a first embedding of each biological interaction profile of the plurality of biological interaction profiles and determine the probability of drug induced liver injury based on the first embedding of each biological interaction profile of the plurality of biological interaction profiles.

5. The method of claim 4, further comprising:
determining, for inclusion in the training dataset, a molecular structure representation for each drug of the plurality of drugs; and
training, based at least on the training dataset, the liver injury prediction model to generate a second embedding of the molecular structure representation of each drug of the plurality of drugs and determine the probability of drug induced liver injury based on the second embedding of the molecular structure representation of each drug of the plurality of drugs.

6. The method of claim 5, wherein the molecular structure representation of each drug of the plurality of drugs comprises an extended-connectivity fingerprint (ECFP).

7. The method of claim 5, further comprising:
determining, for inclusion in the training dataset, one or more molecular properties of each drug of the plurality of drugs; and
training, based at least on the training dataset, the liver injury prediction model to generate a third embedding of the one or more molecular properties of each drug of the plurality of drugs and determine the probability of drug induced liver injury based on the third embedding of the one or more molecular properties of each drug of the plurality of drugs.

8. The method of claim 7, wherein the one or more molecular properties include at least one of a molecular weight, a topological surface area, a partition coefficient (cLogP), and a distribution coefficient (cLogD).

9. The method of claim 1, wherein the plurality of drugs include one or more of a drug known to be positive for drug induced liver injury or a drug known to be negative for drug induced liver injury.

10. The method of claim 1, wherein the trained liver injury prediction model determines the probability of liver injury associated with the drug by at least generating a first embedding of the biological interaction profile of the drug, and determining, based at least on the first embedding of the biological interaction profile of the drug, the probability of the liver injury associated with the drug.

11. The method of claim 10, wherein the trained liver injury prediction model determines the probability of liver injury associated with the drug further based at least on a second embedding of a molecular structure representation of the drug.

12. The method of claim 10, wherein the trained liver injury prediction model determines the probability of liver injury associated with the drug further based at least on a second embedding of one or more molecular properties of the drug.

13. The method of claim 1, further comprising:
generating, based at least on the knowledge graph, the biological interaction profile of the drug, wherein the biological interaction profile of the drug is generated by performing one or more random walks across the knowledge graph, and wherein each random walk of the one or more random walks starts at a node in the knowledge graph corresponding to a protein affected by the drug.

14. The method of claim 13, wherein the biological interaction profile of the drug includes, for each node included in the knowledge graph, a frequency of the node being visited during the one or more random walks across the knowledge graph.

15. The method of claim 1, further comprising:
identifying, based at least on the probability of liver injury associated with the drug, the drug as positive or negative for drug induced liver damage.

16. The method of claim 15, wherein the drug is further identified as causing drug induced liver damage based on one or more in vitro measurements and/or in vivo characterization associated with the drug.

17. The method of claim 16, further comprising:
determining that the one or more in vitro measurements and/or in vivo characterization associated with the drug satisfy a first threshold; and
in response to the one or more in vitro measurements and/or in vivo characterization associated with the drug satisfying the first threshold, determining, based at least on the probability of liver injury associated with the drug satisfying a second threshold, the drug as positive for drug induced liver damage.

18. The method of claim 17, further comprising:
determining that the one or more in vitro measurements and/or in vivo characterization associated with the drug fails to satisfy the first threshold; and
in response to the one or more in vitro measurements and/or in vivo characterization associated with the drug failing to satisfy the first threshold, determining, based at least on the probability of liver injury associated with the drug satisfying a third threshold, the drug as positive for drug induced liver damage.

19. A system comprising: one or more processors; and a non-transitory memory coupled to the processors comprising instructions executable by the processors, the processors operable when executing the instructions to:
determine, based at least on a knowledge graph, a plurality of biological interaction profiles associated with a plurality of drugs, the knowledge graph being representative of a plurality of interactions between the plurality of drugs, a plurality of proteins, and a hierarchy of biological functions, and each biological interaction profile of the plurality of biological interaction profiles representative of one or more effects of a corresponding drug being propagated through one or more protein-protein interactions and biological functions;
train, based at least on a training dataset including the plurality of biological interaction profiles, a liver injury prediction model to determine a probability of drug induced liver injury; and
apply the trained liver injury prediction model to determine, based at least on a biological interaction profile of a drug, a probability of liver injury associated with the drug.

20. One or more computer-readable non-transitory storage media embodying software that is operable when executed to:
determine, based at least on a knowledge graph, a plurality of biological interaction profiles associated with a plurality of drugs, the knowledge graph being representative of a plurality of interactions between the plurality of drugs, a plurality of proteins, and a hierarchy of biological functions, and each biological interaction profile of the plurality of biological interaction profiles representative of one or more effects of a corresponding drug being propagated through one or more protein-protein interactions and biological functions;
train, based at least on a training dataset including the plurality of biological interaction profiles, a liver injury prediction model to determine a probability of drug induced liver injury; and
apply the trained liver injury prediction model to determine, based at least on a biological interaction profile of a drug, a probability of liver injury associated with the drug.
